SEQUENCE LISTING (13 sequences)

SEQ ID NO: 1 - DNA, 4395 bp, Homo sapiens
gcagtgggctctggcggaggtcgggagaactgcagggcgaaggccgccgggggctccgcgggctgcggggggaggcacttgacaccggcccggggagaggaggggccgctgtccctgcggccagtgctggatgcggggacccagcgcagaagcagcgccaggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggcaaagaacagaaggagaccaacatcgaatccatgaaaatggagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtaaaaaaaaaaaaaaaa

SEQ ID NO: 2 - DNA, 3604 bp, Homo sapiens
ccgcccccagccccatcgcccccttcccctcccccaagacgggcagctacttccagagcttcagggccgcggctcacacctgagcgcgactgcagaggggctgcacctggccttatggggatcctggagcgggttgtgagaaggaatgggcgcgtggatcgtagcctgaaagacgagtgtgatacggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtaaaaaaaaaaaaaaaa

SEQ ID NO: 3 - DNA, 4314 bp, Homo sapiens
gagagcagaggacgagccgggacgcggcgccgcggcaccagggcgcgcagccgggccggcccgaccccaccggccatacggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggcaaagaacagaaggagaccaacatcgaatccatgaaaatggagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtaaaaaaaaaaaaaaaa

SEQ ID NO: 4 - DNA, 1808 bp, Homo sapiens
gcagtgggctctggcggaggtcgggagaactgcagggcgaaggccgccgggggctccgcgggctgcggggggaggcacttgacaccggcccggggagaggaggggccgctgtccctgcggccagtgctggatgcggggacccagcgcagaagcagcgccaggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggtgagtcctcagcaccaggggcagcctcttctgggcccaccagcataccctgagagtcagggacttggctctccagcaggtcccaggaaggatggtctgggtcgtggctaaaggtctgcttgccaaggctatggcctggaggctactggctggatgcagcctgcgcatatgttttatttggcccatagagtgttttaaacatttaaaaaattagttgccagtatttaaaaatcaaaaaatttcacataaaaatctggagttttggcttctcatgaaaaaaaaaaaagctagatctggcaacagcgggctttcataacgccaacgattgctagactgggataatggcggtccctccatcgccttctgtggctggttgtgggccttagttttctgcagctctacctggcctgcttactctcccacgtgccatgcagttcctgggggttgctgtatttgtagcccctggcctgggcactcaagggcagcagataccctgtttgcctccctgagtgcagaggtcctgagcccaccctagttgggctgactcaactggaaatttggttgtgacagtggcgtggggagagggctgggtgattgtattctgtgtactgcccagcccaggcctcttcatctggggactttttggcctaaccctggaagcctggaaagttgcccacttttctctttcaggttaagccagcaatttcagggccaaccgagctgtaaacatgttagtaatgaggacaactagcatttgtacagggcttcacagtttacaaagcgctttctcatacattatcacatttgatcctcccagggccctgccaggttgttttgcatatgtgcattttaatttcaaaaagtcttccttccaagcgtgtatgatgaaatgagtaaattgattaattggcgtaacttattttgcatggatccaacctaatgttcatgcaggatagagaacatttccagaatacaaatttccaaacttaaaaaaaaaaaaaaaaaaaaaaaaaaaaaaaaaa

SEQ ID NO: 5 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 6 - protein, 723 aa, Homo sapiens
MGILERVVRRNGRVDRSLKDECDTAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 7 - protein, 166 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGESSAPGAASSGPTSIP

SEQ ID NO: 8 - DNA, 20 bp, Artificial Sequence
  Description of Artificial Sequence Synthetic primer
agcccctgactgatgttctg

SEQ ID NO: 9 - DNA, 20 bp, Artificial Sequence
  Description of Artificial Sequence Synthetic primer
tggaagggcgattggtagac

SEQ ID NO: 10 - DNA, 24 bp, Artificial Sequence
  Description of Artificial Sequence Synthetic primer
ggaggtgcaatggctgtcttgtcc

SEQ ID NO: 11 - DNA, 26 bp, Artificial Sequence
  Description of Artificial Sequence Synthetic primer
ctgccttgggtcaccttacacctcac

SEQ ID NO: 12 - DNA, 22 bp, Artificial Sequence
  Description of Artificial Sequence Synthetic primer
ctctcgggactcctggctccac

SEQ ID NO: 13 - DNA, 22 bp, Artificial Sequence
  Description of Artificial Sequence Synthetic primer
gggtccagcatgtcccacagga

(19) United States
(12) Patent Application Publication
(10) Pub. No.: US 2013/0196865 A1
(43) Pub. Date: Aug. 1, 2013

(54) METHOD FOR SELECTING AN IPS CELL

(71) Applicants: Konrad Hochedlinger, Boston, MA (US); Matthias Stadtfeld, New York, NY (US)

(72) Inventors: Konrad Hochedlinger, Boston, MA (US); Matthias Stadtfeld, New York, NY (US)

(73) Assignee: THE GENERAL HOSPITAL CORPORATION, Boston, MA (US)

(21) Appl. No.: 13/582,553

(22) PCT Filed: Mar. 3, 2011
(86) PCT No.: PCT/US11/27017

§ 371 Date: Apr. 16, 2013

Related U.S. Application Data
(60) Provisional application No. 61/310,118, filed on Mar. 3, 2010.

(51) Int. Cl. C12N 5/071

(52) U.S. Cl. CPC C12N 5/0696
USPC 506/9; 435/325; 435/377; 435/6.11

(57) ABSTRACT

This application relates to a method for selecting an induced pluripotent stem cell (iPS), the method comprising: selecting an iPS cell that expresses a gene in the Dlk1-Dio3 cluster from a population of iPS cells. The method further comprises: comparing the gene expression profile determined for an iPS cell with the gene expression profile determined for an embryonic stem cell; identifying a gene that is differentially expressed in the embryonic stem cell as compared to the iPS cell; and selecting the desired iPS cell from a population of iPS cells.

### CROSS REFERENCE TO RELATED APPLICATIONS

This application claims benefit of priority under 35 U.S.C. §119(e) of the U.S. Provisional Application No. 61/310,118, filed Mar. 3, 2010, the contents of which are incorporated herein by reference in its entirety.

### FIELD OF THE INVENTION

The field of the invention relates to the selection of an iPS cell from a population of iPS cells.

### BACKGROUND

Induced pluripotent stem cells (iPSCs), generated by overexpression of transcription factors such as Oct4, Sox2, Klf4 and c-Myc in somatic cells (K. Takahashi and S. Yamanaka, Cell 126(4): 663 (2006)), enable the derivation of patient-specific pluripotent cell lines to study and potentially treat degenerative diseases. However, the molecular and functional similarities/differences between iPS cells and blastocyst-derived ESCs, the “gold standard” for pluripotent cells, remain unclear. For example, recent studies have reported major mRNA and miRNA expression differences between ESCs and iPSCs in both mouse and human (Chin, M H., et al Cell Stem Cell 5 (1): 111 (2009); Marchetto, M C. et al., PloS one 4 (9):e7076 (2009); Wilson, K D. et al., Stem cells and development 18(5):749 (2009)). At a functional level, many iPSC clones give rise to low-grade chimeras after injection into blastocysts, indicating an incomplete developmental potential of iPSCs compared with ESCs. Conversely, three recent reports claimed the generation of all-iPSC mice, demonstrating that at least some iPSCs are functionally indistinguishable from ESCs (Zhao, X Y et al., Nature (2009); Boland, M J et al., Nature (2009); Kang, L et al., Cell stem cell 5(2):135 (2009)).

### SUMMARY OF THE INVENTION

Methods are provided herein for selecting an iPS cell from a population of iPS cells by measuring the expression level of e.g., a gene in the Dlk1-Dio3 cluster, and selecting for a cell differentially expressing the gene. In one embodiment, a cell selected using the methods described herein has an enhanced differentiation potential compared to a cell lacking expression of the gene (e.g., a gene in the Dlk1-Dio3 cluster). In another embodiment, the iPS cell expressing the identified gene (e.g., a gene in the Dlk1-Dio3 cluster) is more ES cell-like than an iPS cell lacking such expression. Similarly, methods for discarding iPS cells from a population of iPS cells based on a gene expression profile are also provided herein. Also described herein are methods for screening candidate agents that enhance the differentiation potential of iPS cells.

In one aspect described herein, a method is provided for selecting an induced pluripotent stem cell (iPS) comprising: selecting an iPS cell that expresses a gene in the Dlk1-Dio3 cluster from a population of iPS cells.

In one embodiment of this aspect and all other aspects described herein, the gene is Meg3, Rian or Mirg. In another embodiment, the expression of each of genes Meg3, Rian and Mirg are measured.

In one embodiment of this aspect and all other aspects described herein, the induced pluripotent stem cell is a mammalian iPS cell. In one embodiment, the mammalian iPS cell is a human cell. In another embodiment, the mammalian iPS cell is a mouse cell.

In one embodiment, the method further comprises differentiating the iPS cell selected by measuring differential expression of a gene in e.g., the Dlk1-Dio3 cluster.

In another embodiment of this aspect and all other aspects described herein, the iPS cell expressing the identified gene in the Dlk1-Dio3 cluster (e.g., Meg3, Rian, and/or Mirg) has an enhanced differentiation potential compared to an iPS cell lacking expression of the identified gene in the Dlk1-Dio3 cluster.

In another aspect, provided herein is a method for selecting an induced pluripotent stem (iPS) cell from a population of iPS cells comprising: (a) comparing the gene expression profile determined for an iPS cell with the gene expression profile determined for an embryonic stem cell; (b) identifying a gene that is differentially expressed in the embryonic stem cell as compared to the iPS cell; (c) selecting an iPS cell differentially expressing the gene identified in step (b) from a population of iPS cells.

In one embodiment of this aspect and all other aspects described herein, the gene identified in step (b) is upregulated in the iPS cell as compared to the embryonic stem cell.

In another embodiment of this aspect and all other aspects described herein, the gene identified in step (b) is downregulated in the iPS cell as compared to the embryonic stem cell.

In another embodiment, steps (a)-(c) are repeated. In another embodiment, steps (a)-(c) are repeated a plurality of times (e.g., 2, 3, 4, 5, 6, 7, 8, 9, 10, 20, 50, 100 times or more).

In another embodiment, the iPS cell in step (a) is genetically matched to the embryonic stem cell.

In another embodiment, the method further comprises a step of comparing the epigenetic status of the iPS cell in step (a) with the epigenetic status of the embryonic stem cell.

In another embodiment, the gene expression profile is determined using a gene array or RT-PCR.

In one embodiment, the induced pluripotent stem cell is a mammalian iPS cell. In one embodiment, the mammalian iPS cell is a human cell. In another embodiment, the mammalian iPS cell is a mouse cell.

In another embodiment, the method further comprises differentiating the iPS cell selected using the methods described herein.

In another embodiment, the upregulated gene is a gene in the Dlk-Dio3 cluster. In one embodiment, the gene is Meg3, Rian or Mirg. Alternatively, expression of each of genes Meg3, Rian and Mirg are measured.

In another embodiment, the iPS cell expressing a gene in the Dlk1-Dio3 cluster has an enhanced differentiation potential compared to an iPS cell lacking expression of the gene in the Dlk1-Dio3 cluster.

Also described herein is a method for screening for an agent that enhances iPS cell differentiation potential, the method comprising: (a) providing an iPS cell population lacking expression of one or more genes in the Dlk1-Dio3 cluster, (b) contacting the iPS cell population with a candidate agent; (c) measuring the level of expression of the one or more genes in the Dlk1-Dio3 cluster, wherein expression of the one or more genes is indicative that the agent enhances iPS cell differentiation potential.

In one embodiment, the one or more genes is Meg3, Rian or Mirg. In another embodiment, expression of each of genes Meg3, Rian and Mirg are measured.

In one embodiment, the iPS cell in step (a) is genetically matched to the embryonic stem cell.

In another embodiment, the method further comprises a step of comparing the epigenetic status of the iPS cell in step (a) with the epigenetic status of the embryonic stem cell.

In another embodiment, the candidate agent is selected from the group consisting of: a small molecule, an RNAi molecule, a nucleic acid, a protein, a peptide or an antibody. In one embodiment, the candidate agent alters DNA methylation status.

In one embodiment, the induced pluripotent stem cell is a mammalian iPS cell (e.g., mouse or human cell).

Also provided herein are methods for discarding an induced pluripotent stem (iPS) cell from a population of iPS cells, the method comprising: (a) comparing the gene expression profile determined for an iPS cell with the gene expression profile determined for an embryonic stem cell; (b) identifying a gene that is differentially expressed in the iPS cell stem cell compared to the embryonic stem cell; (c) discarding an iPS cell differentially expressing the gene identified in step (b) from a population of iPS cells.

In one embodiment, the gene identified in step (b) is upregulated in the iPS cell as compared to the embryonic stem cell. Alternatively, in another embodiment, the gene identified in step (b) is downregulated in the iPS cell as compared to the embryonic stem cell.

In another embodiment, the discarded iPS cell has a reduced differentiation potential as compared to a non-discarded iPS cell.

In another embodiment, steps (a)-(c) are repeated (e.g., at least once, at least twice, at least three times, at least 4 times, at least 5 times, at least 10 times, at least 20 times or more).

In another embodiment, the iPS cell in step (a) is genetically matched to the embryonic stem cell.

In another embodiment, the method further comprises a step of comparing the epigenetic status of the iPS cell in step (a) with the epigenetic status of the embryonic stem cell.

In another embodiment, the gene expression profile is determined using a gene array or RT-PCR.

In another embodiment, the induced pluripotent stem cell is a mammalian iPS cell (e.g., human cell or murine cell).

### DEFINITIONS

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by the ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers.

The term “re-programming” as used herein refers to the process of altering the differentiated state of a terminally-differentiated somatic cell to a pluripotent phenotype.

By “differentiated primary cell” or “somatic cell” is meant any primary cell that is not, in its native form, pluripotent as that term is defined herein. The term “somatic cell” also encompasses progenitor cells that are multipotent (e.g., produce more than one cell type) but not pluripotent (e.g., can produce cells from all three germ layers). It should be noted that placing many primary cells in culture can lead to some loss of fully differentiated characteristics. However, simply culturing such cells does not, on its own, render them pluripotent. The transition to pluripotency requires a re-programming stimulus beyond the stimuli that lead to partial loss of differentiated character in culture. Re-programmed pluripotent cells (also referred to herein as “induced pluripotent stem cells”) are also characterized by the capacity for extended passaging without loss of growth potential, relative to primary cell parents, which generally have capacity for only a limited number of divisions in culture.

As used herein, the term “induced pluripotent stem cell” or “iPS cell” or “iPSC” refers to a cell that has been reprogrammed from a somatic cell to a more pluripotent phenotype by any means of reprogramming known in the art with the exception of nuclear transfer. Methods for inducing reprogramming of a somatic cell to an iPS cell are provided herein in the Detailed Description. Some non-limiting examples of methods for reprogramming a somatic cell include e.g., expression of stem cell genes such as Oct4, Sox2, Klf4, and Myc, and treatment of cells with a small molecule or combination of small molecules to induce reprogramming.

As used herein, the term “population of iPS cells” refers to a culture comprising at least two iPS cells. While the “population” refers to the iPS cells in the culture, such a culture can further contain other somatic cells in various stages of reprogramming.

As used herein, the term “Dlk1-Dio3 cluster” refers to a cluster of imprinted genes delineated by the delta-like homolog 1 (Dlk1) gene and the type III iodothyronine deiodinase (Dio3) gene located on mouse chromosome 12qF1 or on human chromosome 14q32. Further information on the Dlk1-Dio3 cluster can be found in e.g., da Rocha, S T et al., Trends in Genetics 24(6):306-316 (2008), which is incorporated herein by reference in its entirety. Exemplary genes that are present within the Dlk1-Dio3 cluster include, but are not limited to, Meg3, Rian, and Mirg.

As used herein, the term “mammalian cell” refers to a cell derived from a mammal; non-limiting examples of which include a murine, bovine, simian, porcine, equine, ovine, or human cell.

As used herein, the term “differentially expressed” when used in reference to a gene indicates that the expression of the gene is either upregulated or downregulated in an iPS cell by at least 20% compared to the expression of the same gene in an embryonic stem cell.

As used herein, the term “upregulated” refers to an increased level of expression of a gene in an iPS cell of at least 20% compared to the expression of the gene in an embryonic stem cell. In some embodiments, expression of the gene is increased by at least 30%, at least 40%, at least 50%, at least 60%, at least 70%, at least 80%, at least 90%, at least 95%, at least 99%, at least 1-fold, at least 2-fold, at least 3-fold, at least 4-fold, at least 5-fold, at least 10-fold, at least 20-fold, at least 50-fold, at least 100-fold, at least 1000-fold or higher in the iPS cell compared to expression of the gene in an embryonic stem cell.

As used herein, the term “downregulated” refers to a decrease in expression of a gene in an iPS cell of at least 20% compared to the expression of the gene in an embryonic stem cell. In some embodiments, expression of the gene is decreased by at least 30%, at least 40%, at least 50%, at least 60%, at least 70%, at least 80%, at least 90%, at least 95%, at least 99%, or even 100% (e.g., below detectable levels using e.g., gene array analysis).

As used herein, the term “enhanced differentiation potential” is used to refer to an iPS cell capable of producing a viable all-iPSC mouse using e.g., a tetraploidy (4n) complementation assay as described herein, compared to an iPS cell that cannot produce a viable all-iPSC mouse using the same assay. Alternatively, “enhanced differentiation potential” can be assessed by measuring degree of coat color chimerism when an iPS cell is injected into e.g., diploid blastocysts. In this instance, an iPS cell with “enhanced differentiation potential” exhibits a higher degree of coat chimerism than an iPS cell without enhanced differentiation potential, as described in the Examples section herein. In some embodiments, an iPS cell with enhanced differentiation potential produces pups with at least 60%, at least 70%, at least 80%, at least 90%, at least 95%, at least 99%, or even 100% coat chimerism, whereas an iPS cell lacking enhanced differentiation potential produces pups with less than 50%, less than 40%, less than 30%, less than 20%, or less than 10% coat chimerism.

As used herein, the term “genetically matched” refers to two cells that are obtained from the same donor subject. For example, an embryonic stem cell derived from a subject is genetically matched to an iPS cell derived from a somatic cell of the same subject. The use of genetically matched cells reduces variability in gene expression that is observed among subjects in a population.

A “subject” in the context of the present invention is preferably a mammal. The mammal can be a human, non-human primate, mouse, rat, dog, cat, horse, or cow, but is not limited to these examples.

As used herein, the term “discarding an induced pluripotent stem cell” refers to the removal of iPS cells from a population such that the population is enriched with iPS cells with a desired gene expression profile. In one embodiment, the population is enriched with iPS cells having enhanced differentiation potential. In another embodiment, the population is enriched with iPS cells expressing a gene in the Dlk-Dio3 cluster.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### BRIEF DESCRIPTION OF THE FIGURES

FIG. 1: Aberrant silencing of the Dlk1-Dio3 gene cluster in mouse iPSCs

(a) Strategy for comparing genetically matched ESCs and iPSCs using “reprogrammable mice” harboring a doxycycline-inducible polycistronic reprogramming cassette (OKSM) in the Col1a1 (Collagen) locus. (b) Morphology of Collagen-OKSM ESCs and iPSCs. (c) Unsupervised clustering of four ESC and six iPSC lines based on microarray expression data. (d) Scatterplot of microarray data comparing iPSCs and ESCs with differentially expressed genes highlighted in green (2-fold, p0.05, t-test with Benjamini-Hochberg correction). Heatmaps were produced showing relative expression levels of selected mRNAs in ESCs and iPSCs, covering in addition to Gtl2 and Rian other imprinted genes (Dlk1, Igf2r and H19) and pluripotency-associated transcripts (Nanog, Sox2 and Pou5f1) (data not shown). (e) Schematic representation of mouse chromosome 12 with position of the Dlk1-Dio3 gene cluster highlighted. Maternally-expressed and paternally-expressed transcripts are shown in red and blue, respectively. A heatmap was produced for miRNAs that are differentially expressed between ESCs and iPSCs (2-fold, p0.01, t-test) (data not shown).

FIG. 2: Full developmental potential of Gtlr^{on} iPSCs

Relative expression levels of Gtl2, Rian, other selected imprinted genes (Dlk1, H19 and Igf2r) and pluripotency-associated transcripts (Sox2 and Nanog) in ESCs and iPSCs derived from hematopoietic stem cells (HSC), granulocyte-macrophage progenitors (GMP), granulocytes (Gran), peritoneal fibroblasts (PF) and tail-tip fibroblasts (TTFs), isolated from three individual reprogrammable mice were compared using a heatmap (data not shown). Four iPSC clones expressing ESC-like levels of Gtl2 and Rian were identified (for technical reasons, iPSC clone #18 could not be analyzed by microarray but instead was evaluated by qPCR. See FIG. 5b). (a) Strategy for assessing the developmental potential of iPSC clones by injection into diploid (2n) and tetraploid (4n) blastocysts to produce chimeric or all-iPSC mice, respectively. Images of representative coat color chimeras were analyzed with agouti coloration indicating iPSC origin (data not shown). (b) Coat color chimerism in mice derived from indicated Gtl2^{off} (grey diamonds), Gtl2^{on} iPSC clones (black diamonds) and ESCs (open diamonds). (c) Statistical analysis of coat color chimerism in mice derived form Gtl2^{off} and Gtl2^{on} iPSC clones. (d) Image of two GFP⁺ all-iPSC pups (left panel) and two agouti all-iPSC mice (right). (e) Scatterplot showing intensity levels of all probe sets covered by microarray analysis with those highlighted that were significantly different between 4n complementation-competent iPSCs (clones #19, #44, #47 and #49) and non-4-n complementation-competent iPSCs (clones #18, #20, #45 and #48) (2-fold, p0.05, t-test with Benjamini-Hochberg correction).

FIG. 3: Epigenetic silencing of the Gtl2 locus in iPSCs

Structure of the Dlk1-Dio3 locus with the position of the genomic regions analyzed by pyrosequencing indicated by black bars. (b) Degree of DNA methylation at IG-DMR and Gtl2 DMR in three Gtl2^{off} iPSC clones, three Gtl2^{on} iPSC clones, three ESCs clones (open bars), as well as parental tail-tip fibroblasts (TTFs). The methylation status of the other regions is shown in FIG. 9. (c) Prevalence of activation-associated (aCH3, aCH4 and H3K4me) and repression-associated (H3K27me) chromatin marks at the Gtl2 promoter in two Gtl2^{off} iPSC clones, two Gtl2^{on} iPSCs clones and ESCs. (d) Gtl2 expression levels as measured by qPCR in subclones derived from Gtl2^{off} clone #45 and Gtl2^{on} clone #49 in the absence (upper panel) or presence (lower panel) of doxycycline (dox). (e) Representative brightfield images of iPSCs culture in the absence or presence of all-trans retinoic acid (RA). (f) Expression levels of Gtl2, other imprinted genes (Igf2, Igf2r) and the pluripotency marker Pou5f1 in cells cultured with (+) or without (−) retinoic acid (RA). Note that the two Gtl2^{off} clones fail to activate Gtl2, but show normal expression levels of the other imprinted genes.

FIG. 4: Developmental defects in embryos derived from Gtl^{off} iPSCs

Fluorescence images were obtained for “all-iPSC” E1 1.5 embryos obtained with Gtl2^{on} clone #47 and Gtl2^{off} clone #48, both of which express EGFP from the ubiquitous ROSA26 locus (data not shown). (a) Frequency of dead and living all-iPSC embryos obtained with two Gtl2^{on} and two Gtl2^{off} iPSC clones upon 4n blastocyst injection. Numbers of blastocysts transferred per clone and numbers of embryos recovered are indicated in brackets. (b) Expression of Glt2, Rian, Mirg and the paternally expressed gene Dlk1 in Gtl2^{off} MEFs relative to Gtl2^{on} MEFs (upper panel) as well as in Gtl2^{mKO} MEFs relative to MEFs isolated from wildtype embryos (lower panel). (c) In situ hybridization against Gtl2 mRNA in MEFs derived from all-iPSC embryos generated with either Gtl2^{on} clone #44 or Gtl2^{off} clone #48. (d) Expression levels of Gtl2, Rian, Mirg and Dlk1 in the indicated tissues isolated from all-iPSC embryos made with Gtl2^{off} iPSCs relative to the levels seen in tissues derived from Gtl2^{on} iPSCs. (e) Degree of DNA methylation at the indicated regions in Gtl2^{off}, Gtl2^{on}, Gtl2^{mKO} and wildtype MEFs. (f) Gtl2 expression levels in iPSC lines derived by subcloning Gtl2^{off} clone #45 in the presence of valproic acid (VA). (g) Images of a fully developed stillborn pup (left) and a uterus filled with resorptions (right) derived after 4n blastocyst injections with either VA-10 or the parental iPSC clone #45, respectively.

FIG. 5: qPCR validation of Gtl2 repression in iPSCs.

Expression levels of the maternally expressed 12qF1 genes Gtl2, Rian and Mirg in three iPSCs clones relative to ESC cells. (b) Gtl2 expression levels measured by qPCR in iPSC clones and ESCs. Four iPSC clones with similar expression levels to ESCs are shown. (c) Expression levels of Gtl2 in 18 iPSC clones derived from keratinocytes isolated from two different Collagen-OKSM mice. Note that all of these iPSCs express Gtl2 at significantly lower levels compared to ESCs. (d) Expression levels of Gtl2 in starting cell populations as measured by qPCR as well as in ESCs. HSCs, hematopoietic stem cells; GMPs, granulocyte-macrophage progenitor; Gran., granulocytes; TTFs, tail-tip fibroblasts; MEFs, mouse embryonic fibroblasts.

FIG. 6: Analysis of published array datasets.

Analysis of expression levels of 294 transcripts that were previously reported to be differentially expressed between ESCs and iPSCs using non-genetically matched cells. None of these genes were differentially expressed in Collagen-OKSM ESCs and derivative iPSCs (1.5 fold, p0.05, t-test). (b-e) Expression of the maternally expressed 12qF1 genes Gtl2, Rian and Mirg and pluripotency genes Pou5f1 and Nanog in published microarray datasets containing ESCs and iPSCs. p-values were determined using Student's t-test when replicate samples were available (all datasets except for d). Different starting populations and, in some cases, different combinations of reprogramming factors were used, b) GSE10806; adult mouse neural stem cells transduced with individual retroviral vectors encoding for either Oct4 and Klf4 (2FiPSCs) or Oct4, Klf4, Sox2 and c-myc (4F-iPSCs). c) GSE14012; MEFs transduced with individual retroviral vectors encoding for Oct4, Klf4, Sox2 and c-myc. d) GSE15775; adult bone marrow mononucleated cells transduced with individual retroviral vectors encoding for Oct4, Klf4, Sox2 and c-myc e) E-MEXP-1037; MEFs and TTFs transduced with individual retroviral vectors encoding for Oct4, Klf4, Sox2 and c-myc. Note the consistent downregulation of 12qF1 genes in iPSCs compared to ESCs.

FIG. 7: Confirmation of origin of all-iPSC mice.

PCR was performed to detect three different Simple Sequence Length Polymorphism (SSLP) markers using genomic DNA isolated from 4n complementation-competent iPSC clones and derivative all-iPSC animals. Genomic DNA from BDF1 mice served as a positive control for the presence of host blastocyst-derived cells. Triangles indicate the position of strain-specific bands; open triangle=DBA (blastocyst-specific), grey triangle=129 (iPSC-specific) and black triangle=B6 (present in both blastocysts and iPSCs).

FIG. 8: Analysis of Gtl2 expression in published 4n complementation-competent cell lines.

Expression levels of Gtl2 and Rian and pluripotency markers Pou5f1 and Nanog in R1 ESCs and 4n complemenation-competent iPSCs from GEO microarray dataset GSE17004. No significant differences (p>0.1) were found. (b) Expression levels of Gtl2, Rian and Mirg and pluripotency markers in CL11 ESCs, two 4n complementation-competent iPSC lines (14D-1 and 14D-101) and one non-4-n complementation-competent iPSC line (20D-3) from GEO dataset GSE16295. Note the dramatic decrease of Gtl2 expression in 20D-3 iPSCs compared to the 4n complementation-competent lines and the ESCs.

FIG. 9: DNA methylation analysis of the Dlk1-Dio3 locus.

Structure of the Dlk1-Dio3 locus with the approximate position of the genomic regions analyzed by pyrosequencing indicated by black bars. (b) Degree of DNA methylation at the indicated regions in Gtl2^{off} iPSC clones, Gtl2^{on} iPSC clones, ESCs clones (open bars), as well as parental tail-tip fibroblasts (TTFs).

FIG. 10: Imprinted gene expression after in vitro differentiation.

Expression levels as measured by qPCR of the 12qF1 genes Rian and Dlk1 in undifferentiated (P0) and retinoic acid (RA) treated Gtl2^{off} iPSCs (iPSC #45 and #48), Gtl2^{on} iPSCs (iPSC #47 and #49) and ESCs (dotted line). Gtl2^{off} iPSCs fail to activate expression of the maternally expressed gene Rian, but express high levels of the paternally expressed gene Dlk1 upon differentiation. (b) Expression levels as measured by qPCR of the imprinted genes Mest, Decorin, Phlda2 and Cdkn1c. Note that all cell lines activate these genes to a similar extent.

FIG. 11: Gtl2 expression in nuclear transfer-derived ESCs.

Schematic representation of the derivation of NT-ESCs directly from somatic cells. NT-ESCs generated in this fashion have been shown to be molecularly indistinguishable from blastocyst-derived ESCs and to support the development of “All-ESC” mice. (b) Expression levels of Gtl2, Pou5f1 and Nanog in five blastocyst-derived ESC lines and five ESC lines derived after nuclear transfer (NT) of somatic cell nuclei into enucleated oocytes (“cloning”). The respective donor cell used for NT is indicated. (c) Experimental strategy to test whether nuclear transfer can rescue the defects seen in Gtl2^{off} iPSCs. (d) Microarray heatmap showing expression of the indicated genes in ESCs, iPSCs generated with either adenoviral vectors (Adeno) or the Collagen-OKSM system (#7, #15) and NT-ESC lines derived from the iPSC clones. Note that Gtl2 and Rian remain stably silenced in the NT clones while expression of the imprinted H19 gene shows clone-to-clone variation.

FIG. 12: Chromatin configuration at the Gtl2 promoter after VA rescue.

Prevalence of activation-associated (aCH3 and H3K4me) and repression-associated (H3K27me) chromatin marks at the Gtl2 promoter in Gtl2^{off} iPSC #45, derivative VA-10 and ESC #1.

FIG. 13: Analysis of embryonic tissues derived after 4n blastocyst injections.

Expression of Gtl2, Rian and Dlk1 in head, heart and limb tissue isolated from midgestation embryos obtained after 4n blastocyst injection of Gtl2^{on} iPSCs, Gtl2^{off} iPSCs and rescued iPSCs VA-10. (b) Expression levels of tissue-specific developmental regulators. (c) Expression levels of imprinted genes that have been implicated in abnormal fetal growth.

FIG. 14: Developmental potential of iPSC clone VA-10.

Frequency of dead or living midgestation (E1 1.5) embryos obtained after blastocyst injection of Gtl2^{off} iPSC clone #45 and its VA-rescued derivative clone. (b) Frequency of failed pregnancies (resorptions, lower panel on the left) and completely developed but stillborn embryos (lower panel on the right) recovered after 4n blastocyst injections of the VA-rescued clone and the parental Gtl2^{off} iPSC line #45.

FIG. 15: Effects of Dnmt3 and Vitamin C in IPSCs methylation.

Dnmt3a causes abnormal methylation in iPSCs. (b) Vitamin C prevents abnormal methylation in iPSCs.

### DETAILED DESCRIPTION

Described herein are methods for selecting an iPS cell from a population of iPS cells by measuring the expression level of e.g., a gene in the Dlk1-Dio3 cluster, and selecting for a cell differentially expressing the gene. In one embodiment, a cell selected using the methods described herein has an enhanced differentiation potential compared to a cell lacking expression of the gene (e.g., a gene in the Dlk1-Dio3 cluster). Similarly, methods for discarding iPS cells from a population of iPS cells based on a gene expression profile are also provided herein.

### Cells

Essentially any primary somatic cell type can be used in the preparation of iPS cells. Some non-limiting examples of primary cells include, but are not limited to, fibroblast, epithelial, endothelial, neuronal, adipose, cardiac, skeletal muscle, immune cells, hepatic, splenic, lung, circulating blood cells, gastrointestinal, renal, bone marrow, and pancreatic cells. The cell can be a primary cell isolated from any somatic tissue including, but not limited to brain, liver, lung, gut, stomach, intestine, fat, muscle, uterus, skin, spleen, endocrine organ, bone, etc.

Where the cell is maintained under in vitro conditions, conventional tissue culture conditions and methods can be used, and are known to those of skill in the art. Isolation and culture methods for various cells are well within the abilities of one skilled in the art.

Further, the parental cell can be from any mammalian species, with non-limiting examples including a murine, bovine, simian, porcine, equine, ovine, or human cell. In one embodiment, the cell is a human cell. In an alternate embodiment, the cell is from a non-human organism such as e.g., a non-human mammal. In one embodiment, the parental cell does not express embryonic stem cell (ES) markers, e.g., Nanog mRNA or other ES markers, thus the presence of Nanog mRNA or other ES markers indicates that a cell has been re-programmed. For clarity and simplicity, the description of the methods herein refers to fibroblasts as the parental cells, but it should be understood that all of the methods described herein can be readily applied to other primary parent cell types.

Where a fibroblast is used, the fibroblast, in one embodiment, is flattened and irregularly shaped prior to the re-programming, and does not express Nanog mRNA. The starting fibroblast will preferably not express other embryonic stem cell markers. The expression of ES-cell markers can be measured, for example, by RT-PCR. Alternatively, measurement can be by, for example, immunofluorescence or other immunological detection approach that detects the presence of polypeptides that are characteristic of the ES phenotype.

### Reprogramming

The production of iPS cells is generally achieved by the introduction of nucleic acid sequences encoding stem cell-associated genes into an adult, somatic cell. In general, these nucleic acids are introduced using viral vectors and expression of the gene products results in cells that are morphologically and biochemically similar to pluripotent stem cells (e.g., embryonic stem cells). This process of altering a cell phenotype from a somatic cell phenotype to a stem cell-like phenotype is termed “reprogramming”.

Reprogramming can be achieved by introducing a combination of stem cell-associated genes including, for example Oct3/4 (Pouf51), Sox1, Sox2, Sox3, Sox 15, Sox 18, NANOG, Klf1, Klf2, Klf4, Klf5, c-Myc, 1-Myc, n-Myc and LIN28. In general, successful reprogramming is accomplished by introducing Oct-3/4, a member of the Sox family, a member of the Klf family, and a member of the Myc family to a somatic cell. In one embodiment of the methods described herein, reprogramming is achieved by delivery of Oct-4, Sox2, c-Myc, and Klf4 to a somatic cell (e.g., fibroblast). In one embodiment, the nucleic acid sequences of Oct-4, Sox2, c-MYC, and Klf4 are delivered using a viral vector, such as an adenoviral vector, a lentiviral vector or a retroviral vector.

In one embodiment, the nucleic acid sequences of Oct-4, Sox2, c-MYC, and Klf4 are delivered using an inducible lentiviral vector. Control of expression of re-programming factors can be achieved by contacting a somatic cell having at least one re-programming factor under the control of an inducible promoter, with a regulatory agent (e.g., doxycycline) or other inducing agent. In certain inducible lentiviral vectors, contacting such a cell with a regulatory agent induces expression of the re-programming factors, while withdrawal of the regulatory agent inhibits expression. In other inducible lentiviral vectors, the opposite is true (i.e., the regulatory agent inhibits expression and removal permits expression). The term “induction of expression” refers to the administration or withdrawal of the a regulatory agent (i.e., depending on the lentiviral vector used) and permits expression of at least one reprogramming factor.

It is contemplated herein that induction of expression is only necessary for a certain portion of the re-programming process. While the time necessary for induction of expression will vary with the somatic cell type used, it is generally necessary to detect at least one iPS cell in a culture prior to stopping the induction stimulus. However, it is well within the abilities of one skilled in the art to identify an appropriate time necessary to treat a somatic cell with an induction stimulus. It is contemplated herein that induction of expression may be as short as four hours, or alternatively expression can be induced for the entire reprogramming process, as well as any integer of time in between. For example, induction of expression can be at least 4 hours, at least 5 hours, at least 6 hours, at least 12 hours, at least 24 hours, at least 48 hours, at least 3 days, at least 4 days, at least 5 days, at least 6 days, at least 7 days, at least 8 days, at least 9 days, at least 10 days, at least 11 days, at least 12 days, at least 13 days, at least 14 days, at least 2.5 weeks, at least 3 weeks, at least 3.5 weeks, at least 4 weeks, at least 5 weeks, at least 6 weeks, at least 7 weeks, at least 8 weeks, at least 3 months, or more until a desired level of induction of iPS cells is detected. It is important to note that induction of expression for long periods of time can be detrimental to cell viability and thus it is contemplated herein upon detection of at least one iPS cell in the culture will signal one of skill in the art to stop the induction of expression. In addition, it is further contemplated that induction of expression is stopped at least 1 day prior to using the iPS cells for stem-cell therapy, diagnostics, administration to a subject, or research purposes.

In another embodiment, an iPS cell is reprogrammed by expression of e.g., Oct-4, Sox2, c-MYC, and Klf4 using a non-integrating vector (e.g., adenovirus). While retroviral vectors incorporate into the host cell genome and can potentially disrupt normal gene function, non-integrating vectors have the advantage of controlling expression of a gene product by extra-chromosomal transcription. It follows that since non-integrating vectors do not become part of the host genome, non-integrating vectors tend to express a nucleic acid transiently in a cell population. This is due in part to the fact that the non-integrating vectors are often rendered replication deficient. Thus, non-integrating vectors have several advantages over retroviral vectors including but not limited to: (1) no disruption of the host genome, and (2) transient expression, and (3) no remaining viral integration products. Some non-limiting examples of non-integrating vectors include adenovirus, baculovirus, alphavirus, picornavirus, and vaccinia virus. In one embodiment, the non-integrating viral vector is an adenovirus. The advantages of non-integrating viral vectors further include the ability to produce them in high titers, their stability in vivo, and their efficient infection of host cells.

The viral titer necessary to achieve a desired (i.e., effective) level of gene expression in a host cell is dependent on many factors, including, for example, the cell type, gene product, culture conditions, co-infection with other viral vectors, and co-treatment with other agents, among others. It is well within the abilities of one skilled in the art to test a range of titers for each virus or combination of viruses by detecting the expression levels of either (a) a marker expression product, or (b) a test gene product. Detection of protein expression in cells can be achieved by several techniques including Western blot analysis, immuno-cytochemistry, and fluorescence-mediated detection, among others. It is contemplated that experiments are first optimized by testing a variety of titer ranges for each cell type under the desired culture conditions. Once an optimal titer of a virus or a cocktail of viruses is determined, then that protocol will be used to induce the reprogramming of somatic cells.

In addition to viral titers, it is also important that the infection and induction times are appropriate with respect to different cells. One of skill in the art can test a variety of time points for infection or induction using a viral vector and recover induced pluripotent stem cells from a given somatic cell type.

While it is understood that reprogramming is usually accomplished by viral delivery of stem-cell associated genes, it is also contemplated herein that reprogramming can be induced using other delivery methods (e.g., by treatment of the cells with a small molecule or cocktail of small molecules).

The efficiency of reprogramming (i.e., the number of reprogrammed cells) can be enhanced by the addition of various small molecules as shown by Shi, Y., et al (2008) Cell-Stem Cell 2:525-528, Huangfu, D., et al (2008) Nature Biotechnology 26(7):795-797, Marson, A., et al (2008) Cell-Stem Cell 3:132-135, which are incorporated herein by reference in their entirety. It is contemplated that the methods described herein can also be used in combination with a single small molecule (or a combination of small molecules) that enhances the efficiency of induced pluripotent stem cell production. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), and trichostatin (TSA), among others.

### Confirming Pluripotency and Cell Reprogramming

To confirm the induction of pluripotent stem cells, isolated clones can be tested for the expression of a stem cell marker. Such expression identifies the cells as induced pluripotent stem cells. Stem cell markers can be selected from the non-limiting group including SSEA1, CD9, Nanog, Fbx15, Ecat1, Esg1, Eras, Gdf3, Fgf4, Cripto, Dax1, Zpf296, S1c2a3, Rex1, Utf1, and Nat1. Methods for detecting the expression of such markers can include, for example, RT-PCR and immunological methods that detect the presence of the encoded polypeptides.

The pluripotent stem cell character of the isolated cells can be confirmed by any of a number of tests evaluating the expression of ES markers and the ability to differentiate to cells of each of the three germ layers. As one example, teratoma formation in nude mice can be used to evaluate the pluripotent character of the isolated clones. The cells are introduced to nude mice and histology is performed on a tumor arising from the cells. The growth of a tumor comprising cells from all three germ layers further indicates that the cells are pluripotent stem cells.

### Gene Expression

Gene expression or protein expression can be assessed using methods known in the art including microarrays, transcriptome analysis, proteomics analysis, DNA chips etc. Nucleic acid arrays that are useful in the present invention include, but are not limited to those that are commercially available from Affymetrix (Santa Clara, Calif.). Such methods permit the expressional analysis of gene(s) in the Dlk1-Dio3 cluster.

A gene expression profile can be expressed as a heat map, which in one embodiment can show how experimental conditions influence production of mRNA (e.g., expression) for a set of genes. Typically a heat map summarizes expression levels of a set of genes or subset of genes (e.g., entire genome or Dlk1-Dio3 cluster) and can be compared with another heat map (e.g., derived for a different cell type or for different culture conditions) to indicate genes that are upregulated, downregulated, and genes that are not altered in expression. Such heat map comparisons can be among cells or clonal cell populations to indicate expressional differences between the two cells (e.g., iPS cell vs. ES cell; or iPS cell vs. iPS cell) or among homogenous cell populations. Alternatively, heat maps can be used to compare cells cultured under different conditions, or treated with e.g., small molecules, peptides, antibodies etc as compared to untreated cells to determine changes in gene expression. Variability in gene expression can be minimized by obtaining two cells to be compared from the same donor subject, such that the cells are genetically matched (e.g., highly similar at the genomic level).

Selecting or Discarding Cells from a Population

Essentially any method known in the art can be used to select cells expressing a gene from the Dlk1-Dio3 cluster (or discard cells lacking such expression) from a heterogeneous population containing iPS cells. In one embodiment, the cells expressing a gene in the Dlk1-Dio3 cluster can be selected using flow cytometry (e.g., fluorescence activated cell sorting (FACS)). Alternatively, a population of iPS cells can be enriched for cells expressing a gene in the Dlk1-Dio3 cluster by discarding cells not expressing such a gene using flow cytometry techniques.

Suitable FACS system parameters used to detect and sort fluorescent cells can be determined by one of skill in the art. The excitation and emission maxima for commercially available dyes are known in the art. FACS can be used to select a subpopulation of cells exhibiting higher fluorescence from the population of cells analyzed. The process enables the identification and isolation of cells expressing a gene in the Dlk1-Dio3 cluster. The selected subpopulation of cells can undergo multiple rounds of selection to isolate those cells exhibiting the highest levels of expression.

The selection parameters/criteria used to isolate a desired subpopulation using FACS may vary. Typically, the subpopulation comprises cells exhibiting the highest fluorescence with the total population assayed. In one embodiment, the top 50% of the total population of cells exhibiting fluorescence are selected (i.e. the “subpopulation”), preferably the top 25%, more preferably the top 10%, more preferably the top 5%, even more preferably the top 1%, yet even more preferably the top 0.5%, and most preferably the top 0.1%. Typically, at least 20,000-50,000 events (cells) are analyzed to set up the gates for sorting. The sorted events may vary depending upon the population of the cells available.

In another embodiment, a cell can be selected manually from a population using e.g., a pipette tip. In another embodiment, a cell can be selected using laser-assisted selection of an individual cell. Cells can also be selected by immunocytochemistry techniques, where a cell is treated with e.g., a fluorescent antibody specific for a gene in the Dlk1-Dio3 cluster and the cell is selected based on fluorescence. Cells can also be selected based on e.g., morphology and phenotypic characteristics.

An aspect of the application relates to a method for selecting an induced pluripotent stemcell (iPS), the method comprising: inhibiting methylation in an iPS cell, and selecting an iPS cell that expresses a gene in the Dlk1-Dio3 cluster from a population of iPS cells. In one embodiment, inhibiting methylation is effected by repression of an enzyme. In further embodiment, the enzyme is Dnmt3a. In another embodiment, inhibiting methylation is effected by addition of ascorbic acid to a cell culture medium during reprogramming. In one embodiment, the methylation in an iPS cell is inhibited by at least 10% relative to a standard (e.g., a cell produced without inhibition of methylation or not incubated with a methylation inhibitor). In one embodiment, the methylation in an iPS cell is inhibited by at least 20%, 30%, 40%, 50%, 60%, 70%, 80%, 90%, 95%, 99%, up to and including 100% relative to a standard. In one embodiment, the methylation is de novo methylation. In one embodiment, inhibition of methylation is effected before reprogramming, in any way known to one of skill in the art.

An aspect of the application relates to a method for selecting an induced pluripotent stem cell (iPS), the method comprising: inhibiting DNA methylation during reprogramming of a somatic cell or cell population, wherein the reprogramming generates a population of iPS cells; and selecting an iPS cell from the population of iPS cells that has enhanced differentiation potential relative to an iPS cell generated in the absence of methylation inhibition. In one embodiment, the selected cell expresses a gene in the Dlk1-Dio3 cluster. In one embodiment, the selected cell has decreased methylation of the Gtl2 gene. In one embodiment, selecting an iPS cell comprises selecting an iPS cell that expresses a gene in the Dlk1-Dio3 cluster from the population of iPS cells. In one embodiment, inhibiting DNA methylation is effected by the inhibition of a methylase enzyme. In one embodiment, the DNA methylation in an iPS cell is inhibited by at least 10% relative to a standard. In one embodiment, the methylation in an iPS cell is inhibited by at least 20%, 30%, 40%, 50%, 60%, 70%, 80%, 90%, 95%, 99%, up to and including 100% relative to a standard. In one embodiment, the methylation is de novo methylation. In one embodiment, inhibition of methylation is effected before reprogramming, in any way known to one of skill in the art. In one embodiment, the methylase enzyme is Dnmt3a. In one embodiment, the inhibition of a methylase enzyme comprises inhibition of the expression of the methylase enzyme. In one embodiment, the inhibition of the expression comprises contacting the cell with an RNAi agent that targets the expression of the enzyme. In further embodiment, the inhibition of a methylase enzyme comprises contacting said cell with an antibody or antigen-binding fragment thereof that binds said methylase enzyme.

An aspect of the application relates to a method for selecting an induced pluripotent stem cell (iPS), the method comprising: during reprogramming of a somatic cell or cell population, contacting said cell or said population with ascorbic acid, wherein the reprogramming generates a population of iPS cells; and selecting an iPS cell from the population of iPS cells that has enhanced differentiation potential relative to an iPS cell generated in the absence of ascorbic acid. In one embodiment, the selected cell expresses a gene in the Dlk1-Dio3 cluster. In one embodiment, the selected cell has decreased methylation of the Gtl2 gene. In one embodiment, selecting comprises selecting an iPS cell that expresses a gene in the Dlk1-Dio3 cluster from the population of iPS cells. In one embodiment, ascorbic acid is comprised by culture medium.

An aspect of the application relates to a method for selecting an induced pluripotent stem cell (iPS), the method comprising: inhibiting DNA methylation during reprogramming of a somatic cell or cell population, wherein the reprogramming generates a population of iPS cells; and selecting an iPS cell from the population of iPS cells that expresses a gene in the Dlk1-Dio3 gene cluster, wherein the selected cell has enhanced differentiation potential relative to an iPS cell that does not express a gene in the Dkl1-Dio3 gene cluster. In one embodiment, inhibiting DNA methylation in an iPS cell is effected by the inhibition of a methylase enzyme. In one embodiment, the DNA methylation is inhibited by at least 10% relative to a standard. In one embodiment, the methylation in an iPS is inhibited by at least 20%, 30%, 40%, 50%, 60%, 70%, 80%, 90%, 95%, 99%, up to and including 100% relative to a standard. In one embodiment, the methylation is de novo methylation. In one embodiment, the methylation is de novo methylation. In one embodiment, inhibition of methylation is effected before reprogramming, in any one way known to one of skill in the art.

In one embodiment, wherein the methylase enzyme is Dnmt3a. In one embodiment, inhibition of a methylase enzyme comprises inhibition of the expression of the methylase enzyme. In one embodiment, inhibition of the expression comprises contacting the cell with an RNAi agent that targets the expression of the enzyme. In one embodiment, inhibition of a methylase enzyme comprises contacting said cell with an antibody or antigen-binding fragment thereof that binds said methylase enzyme.

An aspect of the application relates to a method for selecting an induced pluripotent stem cell (iPS), the method comprising: during reprogramming of a somatic cell or cell population, contacting said cell or cell population with ascorbic acid, wherein the reprogramming generates a population of iPS cells; and selecting an iPS cell from the population of iPS cells that expresses a gene in the Dlk1-Dio3 gene cluster, wherein the selected cell has enhanced differentiation potential relative to an iPS cell that does not express a gene in the Dkl1-Dio3 gene cluster. In one embodiment, the selected cell has decreased methylation of the Gtl2 gene. In one embodiment, ascorbic acid is comprised by culture medium.

### Tetraploid Complementation Assay

The tetraploid complementation assay is a technique in biology in which cells of two mammalian embryos are combined to form a new embryo. Normal mammalian somatic cells are diploid (i.e., each chromosome is present in duplicate). First, a tetraploid cell in which every chromosome exists fourfold is produced by taking an embryo at the two-cell stage and fusing the two cells by applying an electrical current. The resulting tetraploid cell will continue to divide, and all daughter cells will also be tetraploid. Such a tetraploid embryo can develop normally to the blastocyst stage and will implant in the wall of the uterus. The tetraploid cells can form the extra-embryonic tissue (placenta etc.), however a proper fetus will rarely develop.

In the tetraploid complementation assay, one now combines such a tetraploid embryo (either at the morula or blastocyst stage) with normal diploid embryonic stem cells (ES) or iPS cells from a different organism. In the case of an embryonic stem cell, the embryo develops normally and the fetus is exclusively derived from the ES cell while the extra-embryonic tissues are exclusively derived from the tetraploid cells.

The tetraploid complementation assay can be used to test an iPS cell's differentiation potential. Only iPS cells with an enhanced differentiation potential are capable of permitting normal development of the embryo, while iPS cells lacking enhanced differentiation potential do not produce a viable embryo.

### Epigenetic Status

As used herein, the term “epigenetics” refers to heritable traits (over rounds of cell division and sometimes transgenerationally) that do not involve changes to the underlying DNA sequence and which are often preserved upon cell division. Exemplary epigenetic processes include paramutation, bookmarking, imprinting, gene silencing, X chromosome inactivation, position effect, transvection, maternal effects, and regulation of histone modifications and heterochromatin.

Molecular biology techniques can be used to assess the epigenetic status of a cell including, but not limited to: chromatin immunoprecipitation (together with its large-scale variants ChIP-on-chip and ChIP-seq), fluorescent in situ hybridization, methylation-sensitive restriction enzymes, DNA adenine methyltransferase identification (DamID) and bisulfite sequencing. In one embodiment, bioinformatic methods such as e.g., computational epigenetics can also be used to assess the epigenetic status of a cell.

DNA methylation is one mechanism involved in the epigenetic regulation of gene expression and can be used to determine the epigenetic status of a DNA region and is described in more detail herein below.

### Determining Methylation Status

As used herein, the term “DNA methylation” refers to the addition of a methyl group to DNA. DNA methylation is present in vertebrates and can have profound effects on gene expression. In general, expression of genes is silenced in methylated regions of DNA.

DNA methylation is essential for normal development and is associated with a number of key processes including genomic imprinting, X-chromosome inactivation, suppression of repetitive elements and carcinogenesis. Without wishing to be bound by theory, DNA methylation can (i) physically impede the binding of transcriptional proteins to the gene and (ii) methylated DNA may be bound by proteins known as methyl-CpG-binding domain proteins (MBDs) that in turn recruit additional proteins to the locus (e.g., histone deacetylases and other chromatin remodelling proteins that can modify histones) thereby forming compact, inactive chromatin (e.g., silenced chromatin)

DNA methylation can be assessed by any method known in art or as described herein. In one embodiment, DNA methylation is determined using Methylation Specific PCR (MSP), based on a chemical reaction of sodium bisulfite with DNA that converts unmethylated cytosines of CpG dinucleotides to uracil or UpG, followed by traditional PCR. In another embodiment, DNA methylation can be determined using the “HELP assay”, which is based on restriction enzymes' differential ability to recognize and cleave methylated and unmethylated CpG DNA sites. In another embodiment, ChIP-on-chip assays are used to assess DNA methylation. ChIP-on-chip technology is based on the ability of commercially prepared antibodies to bind to DNA-methylation associated proteins like MCP2.

In another embodiment, methylated DNA immunoprecipitation (MeDIP), analogous to chromatin immunoprecipitation, immunoprecipitation is used to isolate methylated DNA fragments for input into DNA detection methods such as DNA microarrays (MeDIP-chip) or DNA sequencing (MeDIP-seq).

### Screening Methods

In general, the screening assay(s) described herein are useful for identifying agents that enhance differentiation potential. Typically, such a candidate agent will be tested in a population of iPS cells that has been enriched for cells that lack expression of a gene in the Dlk1-Dio3 cluster (e.g., a cell that is unable to produce a viable all-iPSC mouse using a tetraploid complementation assay) and the expression of the gene is monitored. In general, an increase in the expression of a gene in the Dlk1-Dio3 cluster upon treatment with the candidate agent indicates that the candidate agent enhances differentiation potential of the cell.

In one embodiment, gene expression or protein expression patterns are measured in cells cultured in the presence and/or absence of a candidate agent or test compound. To determine effects of the candidate agent on gene or protein expression, the expression profiles in treated cells can be compared to (i) expression patterns prior to initiating treatment of the cell with the environmental pollutant or drug, or (ii) an untreated culture of cells grown under the same growth and/or culture conditions.

When one compares the transcripts or expression products against the control for increased expression, not all the genes surveyed in the Dlk1-Dio3 cluster need to show an increase in expression. Gene expression or protein expression can be assessed using methods known in the art including microarrays, transcriptome analysis, proteomics analysis, DNA chips etc. Nucleic acid arrays that are useful in the present invention include, but are not limited to those that are commercially available from Affymetrix (Santa Clara, Calif.). In another embodiment, immunocytochemistry or histology techniques can be employed in combination with such a gene array to determine the morphological effects of the test compound on the cells. Alternatively, one of skill in the art can employ fluorescently labeled test compounds to track e.g., increase in expression, optimal dose and length of treatment. It is also contemplated herein that one of skill in the art may utilize other visualization methods for measuring cellular processes including, e.g., luciferase or colorimetric assays.

### Candidate Agents

As used herein the term “agent” refers to any organic or inorganic molecule, including modified and unmodified nucleic acids such as antisense nucleic acids, RNA interference agents such as siRNA, shRNA, or miRNA; peptides, peptidomimetics, receptors, ligands, and antibodies.

Essentially any agent can be tested using the above-described cell culture system including e.g., small molecules, proteins, peptides, nucleic acids, drugs, among others. It is contemplated herein that different doses of each candidate agent are tested using the above-described system.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Small molecule libraries can be obtained commercially and screened for efficacy by one of skill in the art.

### DNMT3A and Inhibition Thereof

DNA (cytosine-5)-methyltransferase 3A is a DNA methylase enzyme, referred to interchangeably herein as DNMT3A or Dnmt3a that in humans is encoded by the DNMT3A gene. (Strictly speaking, the convention “DNMT3A” can refer to the human gene and the italicized version “DNMT3A” can refer to the human protein, and the convention “Dnmt3a” and “Dnmt3a” can refer to the murine gene and protein, respectively; as used herein, however, the terms DNMT3A and Dnmt3a are used interchangeably). The methods described herein can be applied in the context of iPS cells and iPS cell generation for cells of any mammal, including, but not limited to human, mouse, rat, etc. Corresponding DNMT3A genes are known in the art. The enzyme participates in CpG methylation of DNA, an epigenetic modification that is important for embryonic development, imprinting, and X-chromosome inactivation. This gene encodes a DNA methyltransferase that is thought to function in de novo methylation, rather than the maintenance of existing methylated sites. The protein localizes to the cytoplasm and nucleus and its expression is developmentally regulated. Alternative splicing results in multiple transcript variants encoding different isoforms.

As used herein, the term “inhibit” or “inhibition” when used, for example, in reference to methylase or DNMT3A, means the reduction or prevention of DNMT3A activity or the reduction or prevention of DNMT3A gene expression. In one embodiment, the inhibition is in a cell. The reduction in activity or gene expression is at least 10% or more, e.g., at least 20%, at least 30%, at least 40%, at least 50%, at least 60%, at least 70%, at least 80%, at least 90%, at least 95%, at least 99% or more, up to and including 100%, i.e., complete inhibition, as compared to a control or standard, which is activity in the absence of an inhibitor. DNMT3A “inhibition,” as the term is used herein, can also apply to genetic knock out by, e.g., CRE-Lox mediated knock out or other recombination approach.

As used herein, a “DNMT3A inhibitor” is an agent (e.g., small molecule, ligand, nucleic acid or an antibody) which inhibits the activity or the expression of the DNMT3A gene by at least 10%, at least 20%, at least 30%, at least 40%, at least 50%, at least 60%, at least 70%, at least 80%, at least 90%, at least 95%, at least 99% or more, up to and including 100% (no activity) in the presence of a DNMT3A inhibitor relative to activity or expression in the absence of such agent.

In one embodiment, the inhibition of the expression of the DNMT3A gene is by RNA interference or RNAi. An “RNAi” agent is one that induces gene silencing via the RNA-Induced Silencing Complex, or RISC. A “DNMT3A inhibitor” can be double-stranded RNA corresponding to a portion of the DNMT3A transcript or mRNA. One strand of such an inhibitor will be substantially complementary to a portion of the DNMT3A transcript or mRNA, including coding and non-coding sequences.

DNMT3A RNAi agents are known in the art and are commercially available, as are formulations for delivering them to cultured cells. As examples, a DNMT3A-specific RNAi agent can include, e.g., SEQ ID NO. 1, SEQ ID NO. 2, SEQ ID NO. 3, SEQ ID NO. 4 or any derivative or fragment thereof that mediates RISC-mediated inhibition of expression of DNMT3A. Design of nucleotide sequences for RNAi agents capable of reducing expression of DNMT3A will be clear to those skilled in the art and can include, but are not limited to, RNAi, shRNA, miRNA, siRNA, morpholinos and aptamers and can include modified forms or analogs of RNAs. In certain embodiments, such nucleic acid probes would be double-stranded siRNAs such as the products available from Santa Cruz Biotechnology (Santa Cruz, Calif.) as catalog #sc-37757 (human) or sc-37758 (mouse). In other embodiments such nucleic acid probes would be shRNA such as the products available from Santa Cruz Biotechnology in both plasmid and lentiviral vectors as, respectively, sc-37757-SH and sc-37757-V for human DNMT3A and sc-37758-SH and sc-37758-V for murine Dnmt3A. In other embodiments, the DNMT3A inhibitor would be RNAi such as one of the products available from Novus Biologicals (Littleton, Colo.) as catalog #H00001788-R02, H00001788-R01, H00001788-R03, or H00001788-R04. Means of delivering such nucleotide sequences to the target cells, e.g., iPS cells or cells undergoing reprogramming to iPS cells, will also be obvious to those skilled in the art and include but are not limited to, delivery of oligonucleotides themselves, delivery by a vector, or delivery of a mixture comprising the oligonucleotide or vector and at least one other compound. Design and delivery of oligonucleotides are typified but not limited by the methods taught in Verreault, M., et al. Current Gene Therapy 2006, 6, 505-533, Lu, P. Y., et al. Trends in Molecular Medicine 2005, 11, 104-113, Huang, C. et al. Expert Opinion on Therapeutic Targets 2008, 12, 637, Cheema, S. K. et al., Wound Repair and Regeneration 2007, 15, 286, Khurana, B. et al., 2010, 10, 139, Shim, M. S. and Kwon, Y. J. FEBS J, 2010, 277, 4814, Walton, S. P., et al., FEBS J 2010, 277, 4806, Sliva, K. and Schnierle, B. S., Virology Journal 2010, 7, 248, Lares, M. R., et al. Trends in Biotechnology, 28, 570, Rossbach, M. Current Molecular Medicine, 2010, 10, 361, Pfiefer, A. and Lehmann, H. Pharmacology and Therapeutics 2010, 126, 217, Matthais, J. et al. (2003) “Gene Silencing by RNAi in Mammalian Cells” In Ausubel, F. M. et al. (Ed.) Current Protocols in Molecular Biology John Wiley & Sons, Inc.: Hoboken, N.J. These publications are hereby incorporated in their entirety by way of reference.

In one embodiment, the inhibition of the activity of DNMT3A is by contacting with or binding of an antibody that specifically recognizes an epitope of the DNMT3A protein (SEQ ID NO. 5, SEQ ID NO. 6, or SEQ ID NO. 7). In certain embodiments, such antibodies would be one or more of the antibodies to DNMT3A available from Santa Cruz Biotechnology as Catalog #sc-10222, sc-271729, sc-20701, sc-10221, sc-10219, sc-135887, sc-70981, sc-70982, sc-52919, sc-130595, sc-130596, sc-130597, sc-271513, sc-365001, sc-20702, sc-10227, sc-52920, sc-70983, sc-52921, sc-56656, sc-10232, sc-20703, sc-10231, sc-10234, sc-70984, sc-52922, sc-103480, sc-70985, sc-81252, sc-20704, sc-10235, sc-130740, sc-10236, sc-20705, sc-10239, and sc-10241. In other embodiments, such antibodies would be one or more of the antibodies specific for DNMT3A available from Novus Biologicals as catalog #NB100-265, NB120-13888, NBP1-04933, NB100-56521, H00001788-B01P, H00001788-PW1, NB300-720, H00001788-Q01, H00001788-D01P, H0001788-P01, H00001788-D01, and NB100-55782.

The activity of DNMT3A can be determined by a change in at least one measurable marker of DNMT3A activity as known to one of skill in the art. In one embodiment, this would be an assay for DNA methylation, for example, methylation of the Dlk1-Dio3 locus or a gene within such locus, e.g., Gtl-2, as described herein in Example 1 and Example 2.

The level of DNMT3A expression can be determined by any method known in the art, e.g., by western blot analysis of the DNMT3A protein level, or by examination of mRNA levels.

The term “agent” as used in the context of an RNAi agent, for example, refers to any entity which is normally not present or not present at the levels being administered to a cell, tissue or subject. Agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins,

As used herein, the term “antibody” refers to immunoglobulin molecules and antigen-binding portions of immunoglobulin molecules, i.e., molecules that contain an antigen binding site that immunospecifically bind an antigen. The terms also refers to antibodies comprised of two immunoglobulin heavy chains and two immunoglobulin light chains as well as a variety of forms besides antibodies; including, for example, Fv, scFV, Fab, and F(ab)′2 as well as bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference).

A DNMT3A inhibitor can be applied to the media, where it contacts the cell and induces its effects. Alternatively, an inhibitor can be intracellular as a result of introduction of a nucleic acid sequence encoding the agent into the cell and its transcription resulting in the production of the nucleic acid and/or protein environmental stimuli within the cell. In some embodiments, the inhibitor is any chemical, entity or moiety, including without limitation synthetic and naturally-occurring non-proteinaceous entities, that specifically inhibits DNMT3A activity or expression. By “specifically” in this context is meant that the inhibitor inhibits DNMT3A expression or activity to the substantial exclusion of inhibition (as the term is defined herein) of non-methylase enzymes, and preferably to the substantial exclusion of inhibition of other methylase enzymes. In certain embodiments the inhibitor is a small molecule chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, “gene silencing” or “gene silenced” in reference to an activity of an RNAi molecule, for example a siRNA or miRNA refers to a decrease in the mRNA level in a cell for a target gene by at least 10% or more, including, for example, at least 20%, at least 30%, at least 40%, at least 50%, at least 60%, at least 70%, at least 80%, at least 90%, at least 95%, at least 99% or more, up to and including 100% (complete inhibition or silencing) of the mRNA level found in the cell without the presence of the RNA interference agent. In one preferred embodiment, the mRNA levels are decreased by at least 70% or more, e.g., at least 80%, at least 90%, at least 95%, at least 99%, up to and including 100%.

As used herein, the term “RNAi” refers to any type of interfering RNA, including, but not limited to siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of different flanking sequences). The term “RNAi” and “RNA interference” with respect to an agent that inhibits expression of DNMT3A, are used interchangeably herein.

As used herein an “siRNA” refers to a nucleic acid that forms a double stranded RNA, which double stranded RNA has the ability to reduce or inhibit expression of a gene or target gene when the siRNA is present or expressed in the same cell as the target gene, DNMT3A. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

As used herein, the term “complementary” or “complementary base pair” refers to A:T and G:C in DNA and A:U in RNA. Most DNA consists of sequences of nucleotides with only four nitrogenous bases: adenine (A); thymine (T); guanine (G); and cytosine (C). Together these bases form the genetic alphabet, and long ordered sequences of them contain, in coded form, much of the information present in genes. Most RNA also consists of sequences of only four bases. However, in RNA, thymine is replaced by uridine (U).

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA, ribosomal DNA and cDNA. Other suitable nucleic acid molecules are RNA, including mRNA, rRNA and tRNA. The nucleic acid molecule can be naturally occurring, as in genomic DNA, or it may be synthetic, i.e., prepared based up human action, or may be a combination of the two.

The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-β-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

The term “vector”, as used herein, refers to a nucleic acid construct designed for delivery to a host cell or transfer between different host cells. As used herein, a vector can be viral or non-viral.

As used herein, the term “expression vector” refers to a vector that has the ability to incorporate and express heterologous nucleic acid fragments in a cell. An expression vector may comprise additional elements, for example, the expression vector may have two replication systems, thus allowing it to be maintained in two organisms, for example in human cells for expression and in a prokaryotic host for cloning and amplification.

As used herein, the term “heterologous nucleic acid fragments” refers to nucleic acid sequences that are not naturally occurring in that cell.

As used herein, the term “viral vector” refers to a nucleic acid vector construct that includes at least one element of viral origin and has the capacity to be packaged into a viral vector particle. The viral vector can contain the DNMT3A gene or a sequence encoding a dsRNA targeting the DNMT3A gene in place of non-essential viral genes. The vector and/or particle may be utilized for the purpose of transferring any nucleic acids into cells either in vitro or in vivo. Numerous forms of viral vectors are known in the art.

The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

```
SEQ ID NO. 1: (DNMT3A transcript variant 1; NCBI GI: 28559068)
      1 gcagtgggct ctggcggagg tcgggagaac tgcagggcga aggccgccgg gggctccgcg
    61 ggctgcgggg ggaggcactt gacaccggcc cggggagagg aggggccgct gtccctgcgg
  121 ccagtgctgg atgcggggac ccagcgcaga agcagcgcca ggtggagcca tcgaagcccc
  181 cacccacagg ctgacagagg caccgttcac cagagggctc aacaccggga tctatgttta
  241 agttttaact ctcgcctcca aagaccacga taattccttc cccaaagccc agcagccccc
  301 cagccccgcg cagccccagc ctgcctcccg gcgcccagat gcccgccatg ccctccagcg
  361 gccccgggga caccagcagc tctgctgcgg agcgggagga ggaccgaaag gacggagagg
  421 agcaggagga gccgcgtggc aaggaggagc gccaagagcc cagcaccacg gcacggaagg
  481 tggggcggcc tgggaggaag cgcaagcacc ccccggtgga aagcggtgac acgccaaagg
  541 accctgcggt gatctccaag tccccatcca tggcccagga ctcaggcgcc tcagagctat
  601 tacccaatgg ggacttggag aagcggagtg agccccagcc agaggagggg agccctgctg
  661 gggggcagaa gggcggggcc ccagcagagg gagagggtgc agctgagacc ctgcctgaag
  721 cctcaagagc agtggaaaat ggctgctgca cccccaagga gggccgagga gcccctgcag
  781 aagcgggcaa agaacagaag gagaccaaca tcgaatccat gaaaatggag ggctcccggg
  841 gccggctgcg gggtggcttg ggctgggagt ccagcctccg tcagcggccc atgccgaggc
  901 tcaccttcca ggcgggggac ccctactaca tcagcaagcg caagcgggac gagtggctgg
  961 cacgctggaa aagggaggct gagaagaaag ccaaggtcat tgcaggaatg aatgctgtgg
1021 aagaaaacca ggggcccggg gagtctcaga aggtggagga ggccagccct cctgctgtgc
1081 agcagcccac tgaccccgca tcccccactg tggctaccac gcctgagccc gtggggtccg
1141 atgctgggga caagaatgcc accaaagcag gcgatgacga gccagagtac gaggacggcc
1201 ggggctttgg cattggggag ctggtgtggg ggaaactgcg gggcttctcc tggtggccag
1261 gccgcattgt gtcttggtgg atgacgggcc ggagccgagc agctgaaggc acccgctggg
1321 tcatgtggtt cggagacggc aaattctcag tggtgtgtgt tgagaagctg atgccgctga
1381 gctcgttttg cagtgcgttc caccaggcca cgtacaacaa gcagcccatg taccgcaaag
1441 ccatctacga ggtcctgcag gtggccagca gccgcgcggg gaagctgttc ccggtgtgcc
1501 acgacagcga tgagagtgac actgccaagg ccgtggaggt gcagaacaag cccatgattg
1561 aatgggccct ggggggcttc cagccttctg gccctaaggg cctggagcca ccagaagaag
1621 agaagaatcc ctacaaagaa gtgtacacgg acatgtgggt ggaacctgag gcagctgcct
1681 acgcaccacc tccaccagcc aaaaagcccc ggaagagcac agcggagaag cccaaggtca
1741 aggagattat tgatgagcgc acaagagagc ggctggtgta cgaggtgcgg cagaagtgcc
1801 ggaacattga ggacatctgc atctcctgtg ggagcctcaa tgttaccctg gaacaccccc
1861 tcttcgttgg aggaatgtgc caaaactgca agaactgctt tctggagtgt gcgtaccagt
1921 acgacgacga cggctaccag tcctactgca ccatctgctg tgggggccgt gaggtgctca
1981 tgtgcggaaa caacaactgc tgcaggtgct tttgcgtgga gtgtgtggac ctcttggtgg
2041 ggccgggggc tgcccaggca gccattaagg aagacccctg gaactgctac atgtgcgggc
2101 acaagggtac ctacgggctg ctgcggcggc gagaggactg gccctcccgg ctccagatgt
2161 tcttcgctaa taaccacgac caggaatttg accctccaaa ggtttaccca cctgtcccag
2221 ctgagaagag gaagcccatc cgggtgctgt ctctctttga tggaatcgct acagggctcc
2281 tggtgctgaa ggacttgggc attcaggtgg accgctacat tgcctcggag gtgtgtgagg
2341 actccatcac ggtgggcatg gtgcggcacc aggggaagat catgtacgtc ggggacgtcc
2401 gcagcgtcac acagaagcat atccaggagt ggggcccatt cgatctggtg attgggggca
2461 gtccctgcaa tgacctctcc atcgtcaacc ctgctcgcaa gggcctctac gagggcactg
2521 gccggctctt ctttgagttc taccgcctcc tgcatgatgc gcggcccaag gagggagatg
2581 atcgcccctt cttctggctc tttgagaatg tggtggccat gggcgttagt gacaagaggg
2641 acatctcgcg atttctcgag tccaaccctg tgatgattga tgccaaagaa gtgtcagctg
2701 cacacagggc ccgctacttc tggggtaacc ttcccggtat gaacaggccg ttggcatcca
2761 ctgtgaatga taagctggag ctgcaggagt gtctggagca tggcaggata gccaagttca
2821 gcaaagtgag gaccattact acgaggtcaa actccataaa gcagggcaaa gaccagcatt
2881 ttcctgtctt catgaatgag aaagaggaca tcttatggtg cactgaaatg gaaagggtat
2941 ttggtttccc agtccactat actgacgtct ccaacatgag ccgcttggcg aggcagagac
3001 tgctgggccg gtcatggagc gtgccagtca tccgccacct cttcgctccg ctgaaggagt
3061 attttgcgtg tgtgtaaggg acatgggggc aaactgaggt agcgacacaa agttaaacaa
3121 acaaacaaaa aacacaaaac ataataaaac accaagaaca tgaggatgga gagaagtatc
3181 agcacccaga agagaaaaag gaatttaaaa caaaaaccac agaggcggaa ataccggagg
3241 gctttgcctt gcgaaaaggg ttggacatca tctcctgatt tttcaatgtt attcttcagt
3301 cctatttaaa aacaaaacca agctcccttc ccttcctccc ccttcccttt tttttcggtc
3361 agacctttta ttttctactc ttttcagagg ggttttctgt ttgtttgggt tttgtttctt
3421 gctgtgactg aaacaagaag gttattgcag caaaaatcag taacaaaaaa tagtaacaat
3481 accttgcaga ggaaaggtgg gagagaggaa aaaaggaaat tctatagaaa tctatatatt
3541 gggttgtttt tttttttgtt ttttgttttt tttttttggg tttttttttt tactatatat
3601 cttttttttg ttgtctctag cctgatcaga taggagcaca agcaggggac ggaaagagag
3661 agacactcag gcggcagcat tccctcccag ccactgagct gtcgtgccag caccattcct
3721 ggtcacgcaa aacagaaccc agttagcagc agggagacga gaacaccaca caagacattt
3781 ttctacagta tttcaggtgc ctaccacaca ggaaaccttg aagaaaatca gtttctagaa
3841 gccgctgtta cctcttgttt acagtttata tatatatgat agatatgaga tatatatata
3901 aaaggtactg ttaactactg tacaacccga cttcataatg gtgctttcaa acagcgagat
3961 gagtaaaaac atcagcttcc acgttgcctt ctgcgcaaag ggtttcacca aggatggaga
4021 aagggagaca gcttgcagat ggcgcgttct cacggtgggc tcttcccctt ggtttgtaac
4081 gaagtgaagg aggagaactt gggagccagg ttctccctgc caaaaagggg gctagatgag
4141 gtggtcgggc ccgtggacag ctgagagtgg gattcatcca gactcatgca ataacccttt
4201 gattgttttc taaaaggaga ctccctcggc aagatggcag agggtacgga gtcttcaggc
4261 ccagtttctc actttagcca attcgagggc tccttgtggt gggatcagaa ctaatccaga
4321 gtgtgggaaa gtgacagtca aaaccccacc tggagcaaat aaaaaaacat acaaaacgta
4381 aaaaaaaaaa aaaaa
SEQ ID NO. 2: (DNMT3A transcript variant 2; NCBI GI: 28559067)
      1 ccgcccccag ccccatcgcc cccttcccct cccccaagac gggcagctac ttccagagct
    61 tcagggccgc ggctcacacc tgagcgcgac tgcagagggg ctgcacctgg ccttatgggg
  121 atcctggagc gggttgtgag aaggaatggg cgcgtggatc gtagcctgaa agacgagtgt
  181 gatacggctg agaagaaagc caaggtcatt gcaggaatga atgctgtgga agaaaaccag
  241 gggcccgggg agtctcagaa ggtggaggag gccagccctc ctgctgtgca gcagcccact
  301 gaccccgcat cccccactgt ggctaccacg cctgagcccg tggggtccga tgctggggac
  361 aagaatgcca ccaaagcagg cgatgacgag ccagagtacg aggacggccg gggctttggc
  421 attggggagc tggtgtgggg gaaactgcgg ggcttctcct ggtggccagg ccgcattgtg
  481 tcttggtgga tgacgggccg gagccgagca gctgaaggca cccgctgggt catgtggttc
  541 ggagacggca aattctcagt ggtgtgtgtt gagaagctga tgccgctgag ctcgttttgc
  601 agtgcgttcc accaggccac gtacaacaag cagcccatgt accgcaaagc catctacgag
  661 gtcctgcagg tggccagcag ccgcgcgggg aagctgttcc cggtgtgcca cgacagcgat
  721 gagagtgaca ctgccaaggc cgtggaggtg cagaacaagc ccatgattga atgggccctg
  781 gggggcttcc agccttctgg ccctaagggc ctggagccac cagaagaaga gaagaatccc
  841 tacaaagaag tgtacacgga catgtgggtg gaacctgagg cagctgccta cgcaccacct
  901 ccaccagcca aaaagccccg gaagagcaca gcggagaagc ccaaggtcaa ggagattatt
  961 gatgagcgca caagagagcg gctggtgtac gaggtgcggc agaagtgccg gaacattgag
1021 gacatctgca tctcctgtgg gagcctcaat gttaccctgg aacaccccct cttcgttgga
1081 ggaatgtgcc aaaactgcaa gaactgcttt ctggagtgtg cgtaccagta cgacgacgac
1141 ggctaccagt cctactgcac catctgctgt gggggccgtg aggtgctcat gtgcggaaac
1201 aacaactgct gcaggtgctt ttgcgtggag tgtgtggacc tcttggtggg gccgggggct
1261 gcccaggcag ccattaagga agacccctgg aactgctaca tgtgcgggca caagggtacc
1321 tacgggctgc tgcggcggcg agaggactgg ccctcccggc tccagatgtt cttcgctaat
1381 aaccacgacc aggaatttga ccctccaaag gtttacccac ctgtcccagc tgagaagagg
1441 aagcccatcc gggtgctgtc tctctttgat ggaatcgcta cagggctcct ggtgctgaag
1501 gacttgggca ttcaggtgga ccgctacatt gcctcggagg tgtgtgagga ctccatcacg
1561 gtgggcatgg tgcggcacca ggggaagatc atgtacgtcg gggacgtccg cagcgtcaca
1621 cagaagcata tccaggagtg gggcccattc gatctggtga ttgggggcag tccctgcaat
1681 gacctctcca tcgtcaaccc tgctcgcaag ggcctctacg agggcactgg ccggctcttc
1741 tttgagttct accgcctcct gcatgatgcg cggcccaagg agggagatga tcgccccttc
1801 ttctggctct ttgagaatgt ggtggccatg ggcgttagtg acaagaggga catctcgcga
1861 tttctcgagt ccaaccctgt gatgattgat gccaaagaag tgtcagctgc acacagggcc
1921 cgctacttct ggggtaacct tcccggtatg aacaggccgt tggcatccac tgtgaatgat
1981 aagctggagc tgcaggagtg tctggagcat ggcaggatag ccaagttcag caaagtgagg
2041 accattacta cgaggtcaaa ctccataaag cagggcaaag accagcattt tcctgtcttc
2101 atgaatgaga aagaggacat cttatggtgc actgaaatgg aaagggtatt tggtttccca
2161 gtccactata ctgacgtctc caacatgagc cgcttggcga ggcagagact gctgggccgg
2221 tcatggagcg tgccagtcat ccgccacctc ttcgctccgc tgaaggagta ttttgcgtgt
2281 gtgtaaggga catgggggca aactgaggta gcgacacaaa gttaaacaaa caaacaaaaa
2341 acacaaaaca taataaaaca ccaagaacat gaggatggag agaagtatca gcacccagaa
2401 gagaaaaagg aatttaaaac aaaaaccaca gaggcggaaa taccggaggg ctttgccttg
2461 cgaaaagggt tggacatcat ctcctgattt ttcaatgtta ttcttcagtc ctatttaaaa
2521 acaaaaccaa gctcccttcc cttcctcccc cttccctttt ttttcggtca gaccttttat
2581 tttctactct tttcagaggg gttttctgtt tgtttgggtt ttgtttcttg ctgtgactga
2641 aacaagaagg ttattgcagc aaaaatcagt aacaaaaaat agtaacaata ccttgcagag
2701 gaaaggtggg agagaggaaa aaaggaaatt ctatagaaat ctatatattg ggttgttttt
2761 ttttttgttt tttgtttttt ttttttgggt tttttttttt actatatatc ttttttttgt
2821 tgtctctagc ctgatcagat aggagcacaa gcaggggacg gaaagagaga gacactcagg
2881 cggcagcatt ccctcccagc cactgagctg tcgtgccagc accattcctg gtcacgcaaa
2941 acagaaccca gttagcagca gggagacgag aacaccacac aagacatttt tctacagtat
3001 ttcaggtgcc taccacacag gaaaccttga agaaaatcag tttctagaag ccgctgttac
3061 ctcttgttta cagtttatat atatatgata gatatgagat atatatataa aaggtactgt
3121 taactactgt acaacccgac ttcataatgg tgctttcaaa cagcgagatg agtaaaaaca
3181 tcagcttcca cgttgccttc tgcgcaaagg gtttcaccaa ggatggagaa agggagacag
3241 cttgcagatg gcgcgttctc acggtgggct cttccccttg gtttgtaacg aagtgaagga
3301 ggagaacttg ggagccaggt tctccctgcc aaaaaggggg ctagatgagg tggtcgggcc
3361 cgtggacagc tgagagtggg attcatccag actcatgcaa taaccctttg attgttttct
3421 aaaaggagac tccctcggca agatggcaga gggtacggag tcttcaggcc cagtttctca
3481 ctttagccaa ttcgagggct ccttgtggtg ggatcagaac taatccagag tgtgggaaag
3541 tgacagtcaa aaccccacct ggagcaaata aaaaaacata caaaacgtaa aaaaaaaaaa
3601 aaaa
SEQ ID NO. 3: (DNMT3A transcript variant 3; NCBI GI: 28559066)
      1 gagagcagag gacgagccgg gacgcggcgc cgcggcacca gggcgcgcag ccgggccggc
    61 ccgaccccac cggccatacg gtggagccat cgaagccccc acccacaggc tgacagaggc
  121 accgttcacc agagggctca acaccgggat ctatgtttaa gttttaactc tcgcctccaa
  181 agaccacgat aattccttcc ccaaagccca gcagcccccc agccccgcgc agccccagcc
  241 tgcctcccgg cgcccagatg cccgccatgc cctccagcgg ccccggggac accagcagct
  301 ctgctgcgga gcgggaggag gaccgaaagg acggagagga gcaggaggag ccgcgtggca
  361 aggaggagcg ccaagagccc agcaccacgg cacggaaggt ggggcggcct gggaggaagc
  421 gcaagcaccc cccggtggaa agcggtgaca cgccaaagga ccctgcggtg atctccaagt
  481 ccccatccat ggcccaggac tcaggcgcct cagagctatt acccaatggg gacttggaga
  541 agcggagtga gccccagcca gaggagggga gccctgctgg ggggcagaag ggcggggccc
  601 cagcagaggg agagggtgca gctgagaccc tgcctgaagc ctcaagagca gtggaaaatg
  661 gctgctgcac ccccaaggag ggccgaggag cccctgcaga agcgggcaaa gaacagaagg
  721 agaccaacat cgaatccatg aaaatggagg gctcccgggg ccggctgcgg ggtggcttgg
  781 gctgggagtc cagcctccgt cagcggccca tgccgaggct caccttccag gcgggggacc
  841 cctactacat cagcaagcgc aagcgggacg agtggctggc acgctggaaa agggaggctg
  901 agaagaaagc caaggtcatt gcaggaatga atgctgtgga agaaaaccag gggcccgggg
  961 agtctcagaa ggtggaggag gccagccctc ctgctgtgca gcagcccact gaccccgcat
1021 cccccactgt ggctaccacg cctgagcccg tggggtccga tgctggggac aagaatgcca
1081 ccaaagcagg cgatgacgag ccagagtacg aggacggccg gggctttggc attggggagc
1141 tggtgtgggg gaaactgcgg ggcttctcct ggtggccagg ccgcattgtg tcttggtgga
1201 tgacgggccg gagccgagca gctgaaggca cccgctgggt catgtggttc ggagacggca
1261 aattctcagt ggtgtgtgtt gagaagctga tgccgctgag ctcgttttgc agtgcgttcc
1321 accaggccac gtacaacaag cagcccatgt accgcaaagc catctacgag gtcctgcagg
1381 tggccagcag ccgcgcgggg aagctgttcc cggtgtgcca cgacagcgat gagagtgaca
1441 ctgccaaggc cgtggaggtg cagaacaagc ccatgattga atgggccctg gggggcttcc
1501 agccttctgg ccctaagggc ctggagccac cagaagaaga gaagaatccc tacaaagaag
1561 tgtacacgga catgtgggtg gaacctgagg cagctgccta cgcaccacct ccaccagcca
1621 aaaagccccg gaagagcaca gcggagaagc ccaaggtcaa ggagattatt gatgagcgca
1681 caagagagcg gctggtgtac gaggtgcggc agaagtgccg gaacattgag gacatctgca
1741 tctcctgtgg gagcctcaat gttaccctgg aacaccccct cttcgttgga ggaatgtgcc
1801 aaaactgcaa gaactgcttt ctggagtgtg cgtaccagta cgacgacgac ggctaccagt
1861 cctactgcac catctgctgt gggggccgtg aggtgctcat gtgcggaaac aacaactgct
1921 gcaggtgctt ttgcgtggag tgtgtggacc tcttggtggg gccgggggct gcccaggcag
1981 ccattaagga agacccctgg aactgctaca tgtgcgggca caagggtacc tacgggctgc
2041 tgcggcggcg agaggactgg ccctcccggc tccagatgtt cttcgctaat aaccacgacc
2101 aggaatttga ccctccaaag gtttacccac ctgtcccagc tgagaagagg aagcccatcc
2161 gggtgctgtc tctctttgat ggaatcgcta cagggctcct ggtgctgaag gacttgggca
2221 ttcaggtgga ccgctacatt gcctcggagg tgtgtgagga ctccatcacg gtgggcatgg
2281 tgcggcacca ggggaagatc atgtacgtcg gggacgtccg cagcgtcaca cagaagcata
2341 tccaggagtg gggcccattc gatctggtga ttgggggcag tccctgcaat gacctctcca
2401 tcgtcaaccc tgctcgcaag ggcctctacg agggcactgg ccggctcttc tttgagttct
2461 accgcctcct gcatgatgcg cggcccaagg agggagatga tcgccccttc ttctggctct
2521 ttgagaatgt ggtggccatg ggcgttagtg acaagaggga catctcgcga tttctcgagt
2581 ccaaccctgt gatgattgat gccaaagaag tgtcagctgc acacagggcc cgctacttct
2641 ggggtaacct tcccggtatg aacaggccgt tggcatccac tgtgaatgat aagctggagc
2701 tgcaggagtg tctggagcat ggcaggatag ccaagttcag caaagtgagg accattacta
2761 cgaggtcaaa ctccataaag cagggcaaag accagcattt tcctgtcttc atgaatgaga
2821 aagaggacat cttatggtgc actgaaatgg aaagggtatt tggtttccca gtccactata
2881 ctgacgtctc caacatgagc cgcttggcga ggcagagact gctgggccgg tcatggagcg
2941 tgccagtcat ccgccacctc ttcgctccgc tgaaggagta ttttgcgtgt gtgtaaggga
3001 catgggggca aactgaggta gcgacacaaa gttaaacaaa caaacaaaaa acacaaaaca
3061 taataaaaca ccaagaacat gaggatggag agaagtatca gcacccagaa gagaaaaagg
3121 aatttaaaac aaaaaccaca gaggcggaaa taccggaggg ctttgccttg cgaaaagggt
3181 tggacatcat ctcctgattt ttcaatgtta ttcttcagtc ctatttaaaa acaaaaccaa
3241 gctcccttcc cttcctcccc cttccctttt ttttcggtca gaccttttat tttctactct
3301 tttcagaggg gttttctgtt tgtttgggtt ttgtttcttg ctgtgactga aacaagaagg
3361 ttattgcagc aaaaatcagt aacaaaaaat agtaacaata ccttgcagag gaaaggtggg
3421 agagaggaaa aaaggaaatt ctatagaaat ctatatattg ggttgttttt ttttttgttt
3481 tttgtttttt ttttttgggt tttttttttt actatatatc ttttttttgt tgtctctagc
3541 ctgatcagat aggagcacaa gcaggggacg gaaagagaga gacactcagg cggcagcatt
3601 ccctcccagc cactgagctg tcgtgccagc accattcctg gtcacgcaaa acagaaccca
3661 gttagcagca gggagacgag aacaccacac aagacatttt tctacagtat ttcaggtgcc
3721 taccacacag gaaaccttga agaaaatcag tttctagaag ccgctgttac ctcttgttta
3781 cagtttatat atatatgata gatatgagat atatatataa aaggtactgt taactactgt
3841 acaacccgac ttcataatgg tgctttcaaa cagcgagatg agtaaaaaca tcagcttcca
3901 cgttgccttc tgcgcaaagg gtttcaccaa ggatggagaa agggagacag cttgcagatg
3961 gcgcgttctc acggtgggct cttccccttg gtttgtaacg aagtgaagga ggagaacttg
4021 ggagccaggt tctccctgcc aaaaaggggg ctagatgagg tggtcgggcc cgtggacagc
4081 tgagagtggg attcatccag actcatgcaa taaccctttg attgttttct aaaaggagac
4141 tccctcggca agatggcaga gggtacggag tcttcaggcc cagtttctca ctttagccaa
4201 ttcgagggct ccttgtggtg ggatcagaac taatccagag tgtgggaaag tgacagtcaa
4261 aaccccacct ggagcaaata aaaaaacata caaaacgtaa aaaaaaaaaa aaaa
SEQ ID NO. 4: (DNMT3A transcript variant 4; NCBI GI: 28559070)
      1 gcagtgggct ctggcggagg tcgggagaac tgcagggcga aggccgccgg gggctccgcg
    61 ggctgcgggg ggaggcactt gacaccggcc cggggagagg aggggccgct gtccctgcgg
  121 ccagtgctgg atgcggggac ccagcgcaga agcagcgcca ggtggagcca tcgaagcccc
  181 cacccacagg ctgacagagg caccgttcac cagagggctc aacaccggga tctatgttta
  241 agttttaact ctcgcctcca aagaccacga taattccttc cccaaagccc agcagccccc
  301 cagccccgcg cagccccagc ctgcctcccg gcgcccagat gcccgccatg ccctccagcg
  361 gccccgggga caccagcagc tctgctgcgg agcgggagga ggaccgaaag gacggagagg
  421 agcaggagga gccgcgtggc aaggaggagc gccaagagcc cagcaccacg gcacggaagg
  481 tggggcggcc tgggaggaag cgcaagcacc ccccggtgga aagcggtgac acgccaaagg
  541 accctgcggt gatctccaag tccccatcca tggcccagga ctcaggcgcc tcagagctat
  601 tacccaatgg ggacttggag aagcggagtg agccccagcc agaggagggg agccctgctg
  661 gggggcagaa gggcggggcc ccagcagagg gagagggtgc agctgagacc ctgcctgaag
  721 cctcaagagc agtggaaaat ggctgctgca cccccaagga gggccgagga gcccctgcag
  781 aagcgggtga gtcctcagca ccaggggcag cctcttctgg gcccaccagc ataccctgag
  841 agtcagggac ttggctctcc agcaggtccc aggaaggatg gtctgggtcg tggctaaagg
  901 tctgcttgcc aaggctatgg cctggaggct actggctgga tgcagcctgc gcatatgttt
  961 tatttggccc atagagtgtt ttaaacattt aaaaaattag ttgccagtat ttaaaaatca
1021 aaaaatttca cataaaaatc tggagttttg gcttctcatg aaaaaaaaaa aagctagatc
1081 tggcaacagc gggctttcat aacgccaacg attgctagac tgggataatg gcggtccctc
1141 catcgccttc tgtggctggt tgtgggcctt agttttctgc agctctacct ggcctgctta
1201 ctctcccacg tgccatgcag ttcctggggg ttgctgtatt tgtagcccct ggcctgggca
1261 ctcaagggca gcagataccc tgtttgcctc cctgagtgca gaggtcctga gcccacccta
1321 gttgggctga ctcaactgga aatttggttg tgacagtggc gtggggagag ggctgggtga
1381 ttgtattctg tgtactgccc agcccaggcc tcttcatctg gggacttttt ggcctaaccc
1441 tggaagcctg gaaagttgcc cacttttctc tttcaggtta agccagcaat ttcagggcca
1501 accgagctgt aaacatgtta gtaatgagga caactagcat ttgtacaggg cttcacagtt
1561 tacaaagcgc tttctcatac attatcacat ttgatcctcc cagggccctg ccaggttgtt
1621 ttgcatatgt gcattttaat ttcaaaaagt cttccttcca agcgtgtatg atgaaatgag
1681 taaattgatt aattggcgta acttattttg catggatcca acctaatgtt catgcaggat
1741 agagaacatt tccagaatac aaatttccaa acttaaaaaa aaaaaaaaaa aaaaaaaaaa
1801 aaaaaaaa
SEQ ID NO. 5: (DNMT3A isoform a; NCBI GI: 28559069)
      1 mpampssgpg dtsssaaere edrkdgeeqe eprgkeerqe psttarkvgr pgrkrkhppv
    61 esgdtpkdpa viskspsmaq dsgasellpn gdlekrsepq peegspaggq kggapaegeg
  121 aaetlpeasr avengcctpk egrgapaeag keqketnies mkmegsrgrl rgglgwessl
  181 rqrpmprltf qagdpyyisk rkrdewlarw kreaekkakv iagmnaveen qgpgesqkve
  241 easppavqqp tdpasptvat tpepvgsdag dknatkagdd epeyedgrgf gigelvwgkl
  301 rgfswwpgri vswwmtgrsr aaegtrwvmw fgdgkfsvvc veklmplssf csafhqatyn
  361 kqpmyrkaiy evlqvassra gklfpvchds desdtakave vqnkpmiewa lggfqpsgpk
  421 gleppeeekn pykevytdmw vepeaaayap pppakkprks taekpkvkei idertrerlv
  481 yevrqkcrni ediciscgsl nvtlehplfv ggmcqncknc flecayqydd dgyqsyctic
  541 cggrevlmcg nnnccrcfcv ecvdllvgpg aaqaaikedp wncymcghkg tygllrrred
  601 wpsrlqmffa nnhdqefdpp kvyppvpaek rkpirvlslf dgiatgllvl kdlgiqvdry
  661 iasevcedsi tvgmvrhqgk imyvgdvrsv tqkhiqewgp fdlviggspc ndlsivnpar
  721 kglyegtgrl ffefyrllhd arpkegddrp ffwlfenvva mgvsdkrdis rflesnpvmi
  781 dakevsaahr aryfwgnlpg mnrplastvn dklelqecle hgriakfskv rtittrsnsi
  841 kqgkdqhfpv fmnekedilw ctemervfgf pvhytdvsnm srlarqrllg rswsvpvirh
  901 lfaplkeyfa cv
SEQ ID NO. 6: (DNMT3A isoform b; NCBI GI: 77176455)
      1 mgilervvrr ngrvdrslkd ecdtaekkak viagmnavee nqgpgesqkv eeasppavqq
    61 ptdpasptva ttpepvgsda gdknatkagd depeyedgrg fgigelvwgk lrgfswwpgr
  121 ivswwmtgrs raaegtrwvm wfgdgkfsvv cveklmplss fcsafhqaty nkqpmyrkai
  181 yevlqvassr agklfpvchd sdesdtakav evqnkpmiew alggfqpsgp kgleppeeek
  241 npykevytdm wvepeaaaya ppppakkprk staekpkvke iidertrerl vyevrqkcrn
  301 iediciscgs lnvtlehplf vggmcqnckn cflecayqyd ddgyqsycti ccggrevlmc
  361 gnnnccrcfc vecvdllvgp gaaqaaiked pwncymcghk gtygllrrre dwpsrlqmff
  421 annhdqefdp pkvyppvpae krkpirvlsl fdgiatgllv lkdlgiqvdr yiasevceds
  481 itvgmvrhqg kimyvgdvrs vtqkhiqewg pfdlviggsp cndlsivnpa rkglyegtgr
  541 lffefyrllh darpkegddr pffwlfenvv amgvsdkrdi srflesnpvm idakevsaah
  601 raryfwgnlp gmnrplastv ndklelqecl ehgriakfsk vrtittrsns ikqgkdqhfp
  661 vfmnekedil wctemervfg fpvhytdvsn msrlarqrll grswsvpvir hlfaplkeyf
  721 acv
SEQ ID NO. 7: (DNMT3A isoform c; NCBI GI: 28559071)
      1 mpampssgpg dtsssaaere edrkdgeeqe eprgkeerqe psttarkvgr pgrkrkhppv
    61 esgdtpkdpa viskspsmaq dsgasellpn gdlekrsepq peegspaggq kggapaegeg
  121 aaetlpeasr avengcctpk egrgapaeag essapgaass gptsip
```

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

Induced pluripotent stem cells (iPSCs) have been generated by enforced expression of defined sets of transcription factors in somatic cells. The molecular and functional similarities/differences between iPS cells and blastocyst-derived embryonic stem cells (ESCs) are now emerging. By comparing genetically identical mouse ESCs and iPSCs, the authors show herein that the overall mRNA and miRNA expression patterns of these cell types are indistinguishable with the exception of a few transcripts and miRNAs encoded on mouse chromosome 12qF1. Specifically, maternally expressed imprinted genes in the Dild-Dio3 cluster including Meg3/Gtl2, Rian and Mirg as well as a larger number of miRNAs encoded within this region were aberrantly silenced in the majority of iPSC clones, irrespective of their cell type of origin. Consistent with a developmental role of the Dlk1-Dio3 gene cluster, iPSC clones with repressed Meg3/Gtl2 contributed poorly to chimeras and failed to support the development of entirely iPSC-derived animals (“all-iPSC mice”). In contrast, iPSC clones with normal expression levels of these genes contributed to high-grade chimeras and generated viable all-iPSC mice. Importantly, treatment of an iPSC clone that had silenced Dlk1-Dio3 and failed to give rise to all-iPSC animals with a histone deacetylase inhibitor reactivated the locus and rescued its ability to support full-term development of exclusively iPSC-derived mice. Thus, the expression state of a single imprinted gene cluster distinguishes most murine iPSCs from ESCs and allows for the prospective identification of iPSC clones that have the full development potential of ESCs.

### Example 1

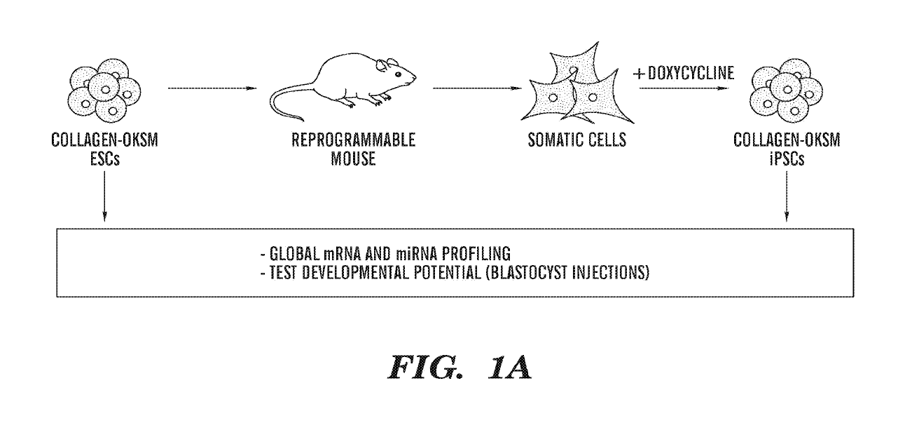

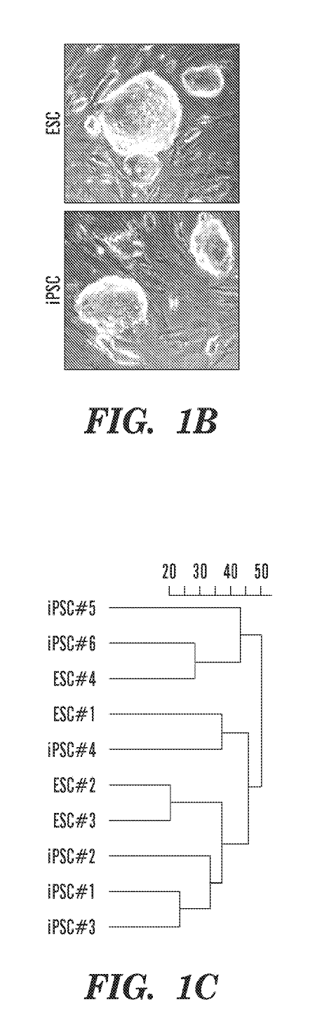

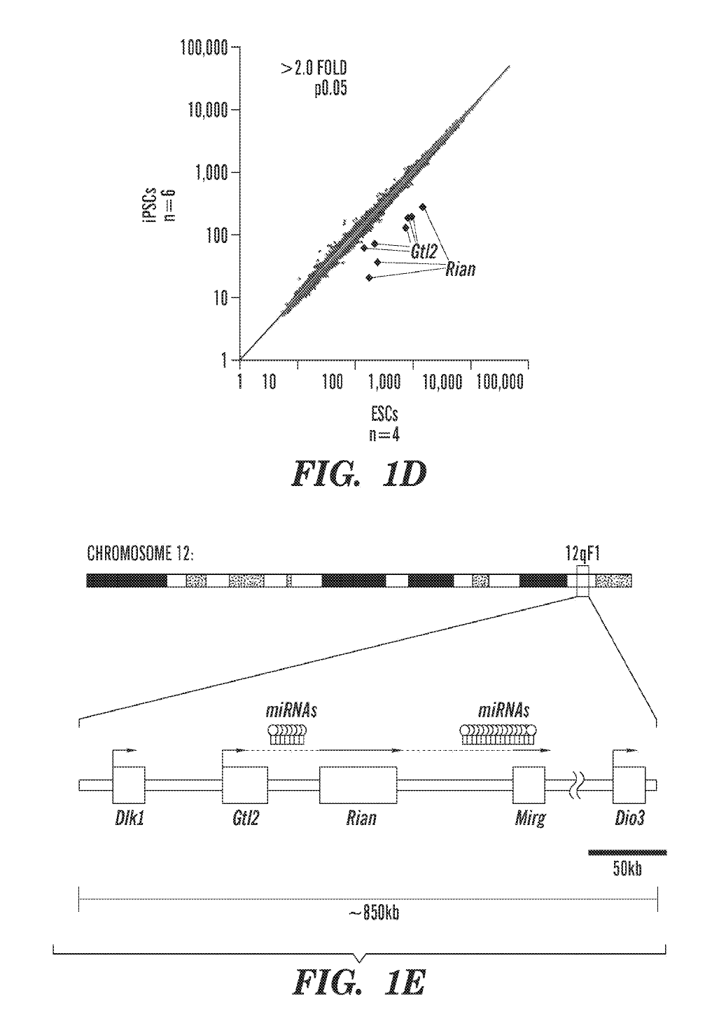

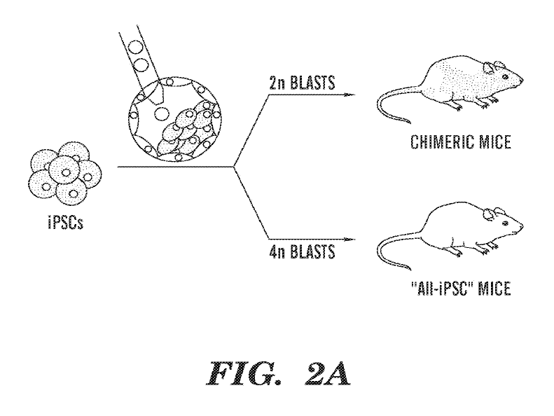

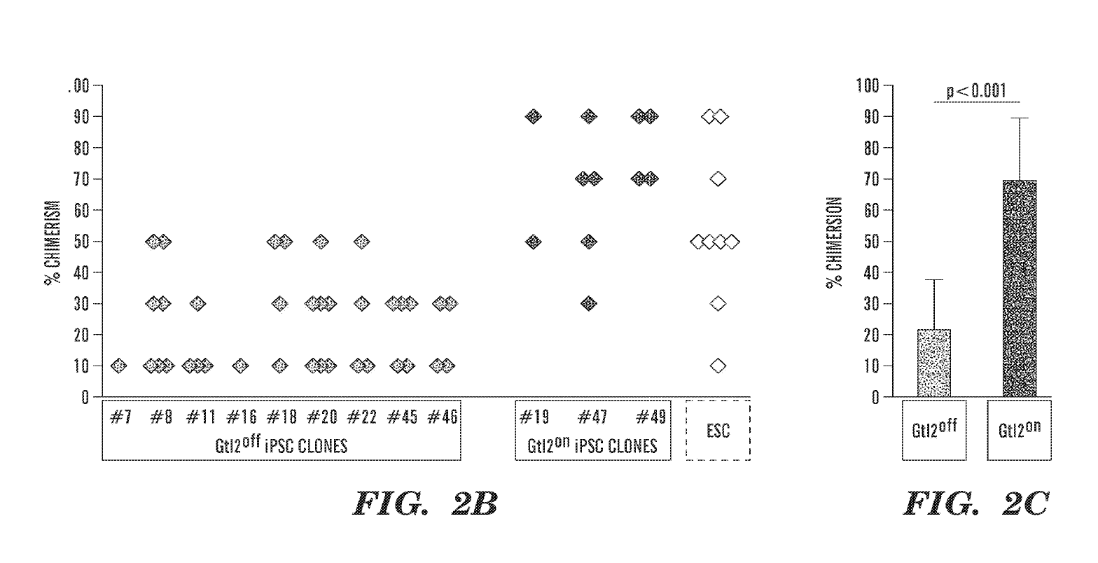

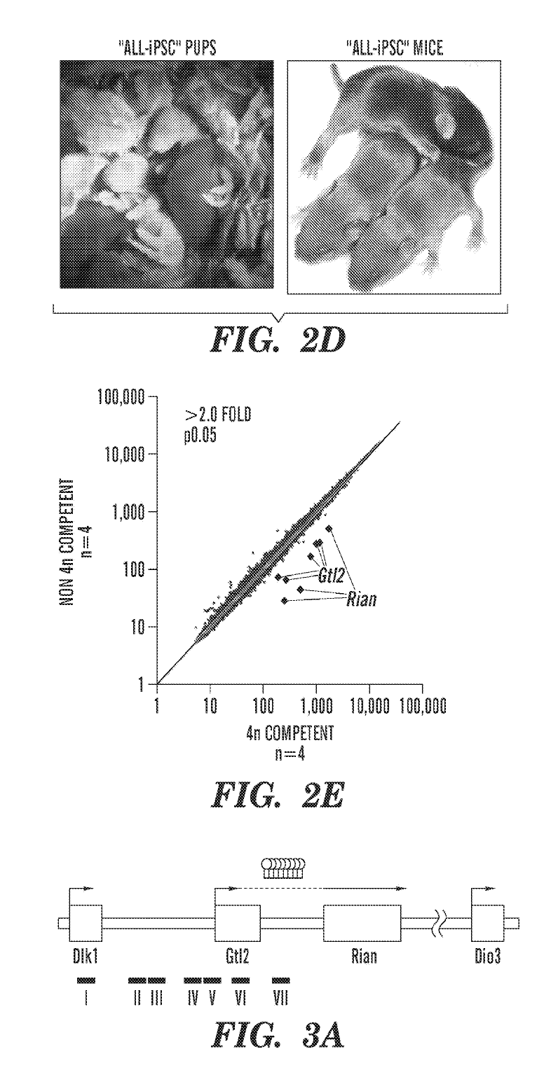

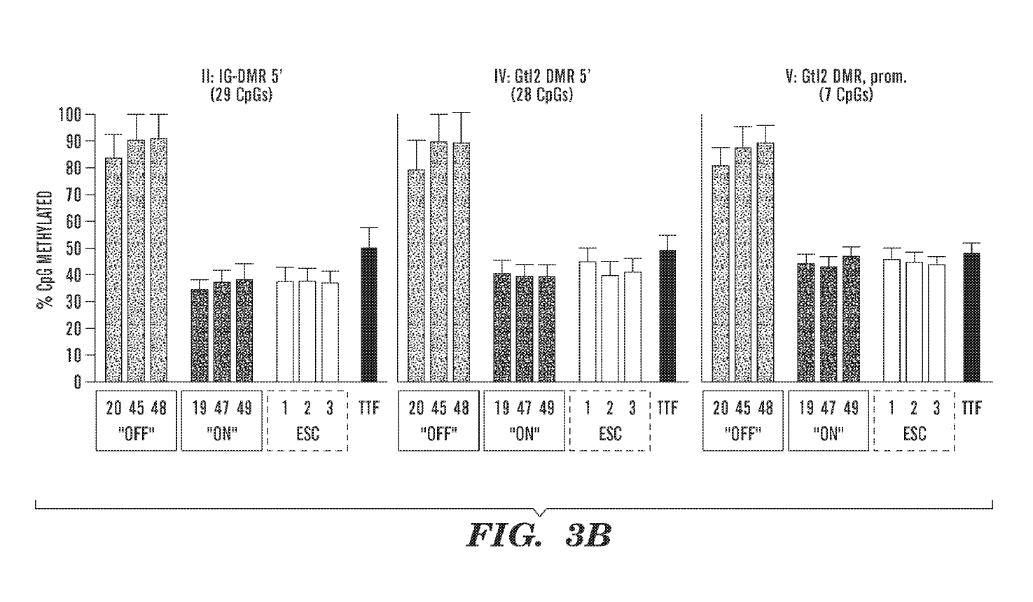

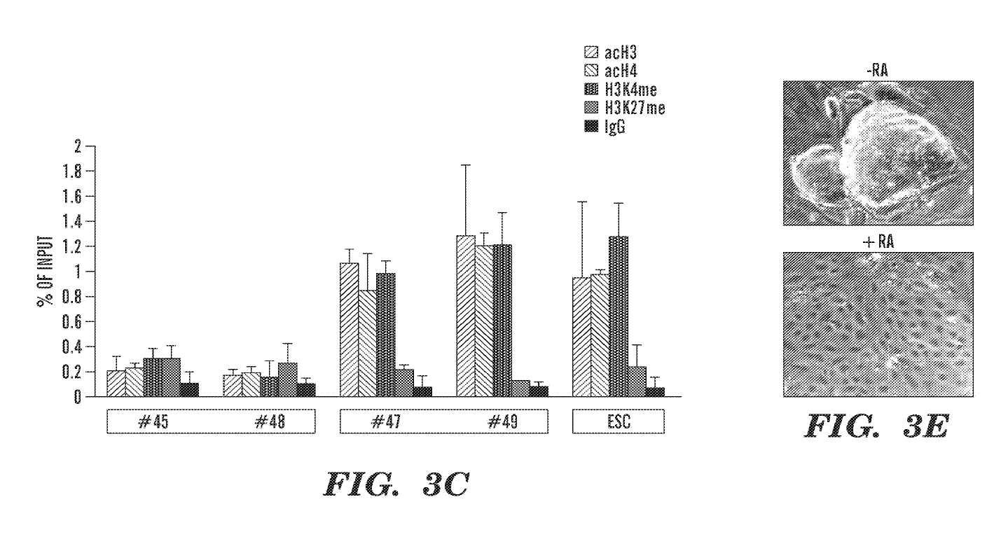

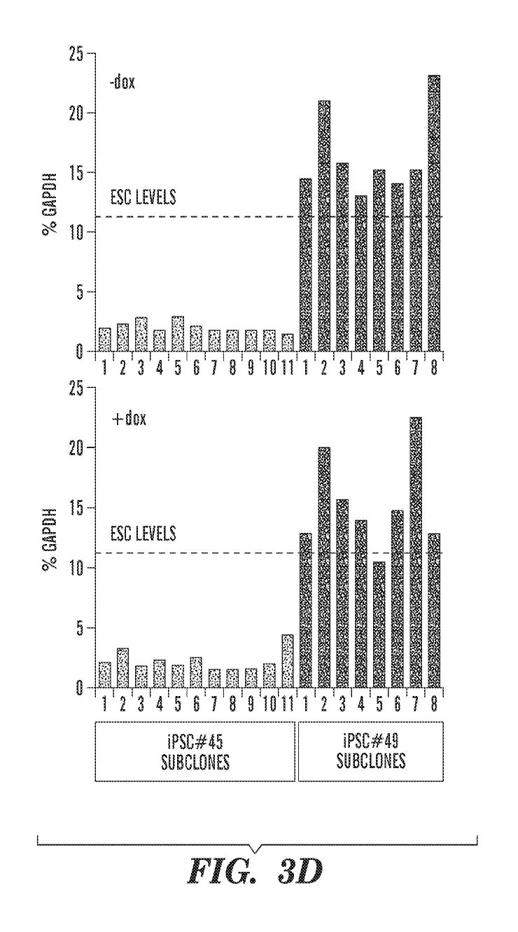

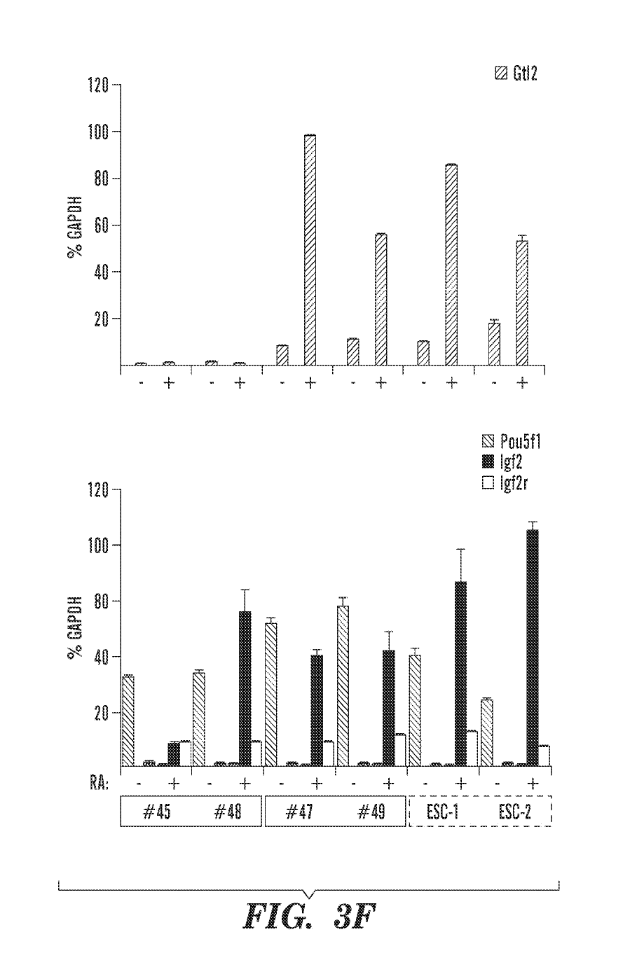

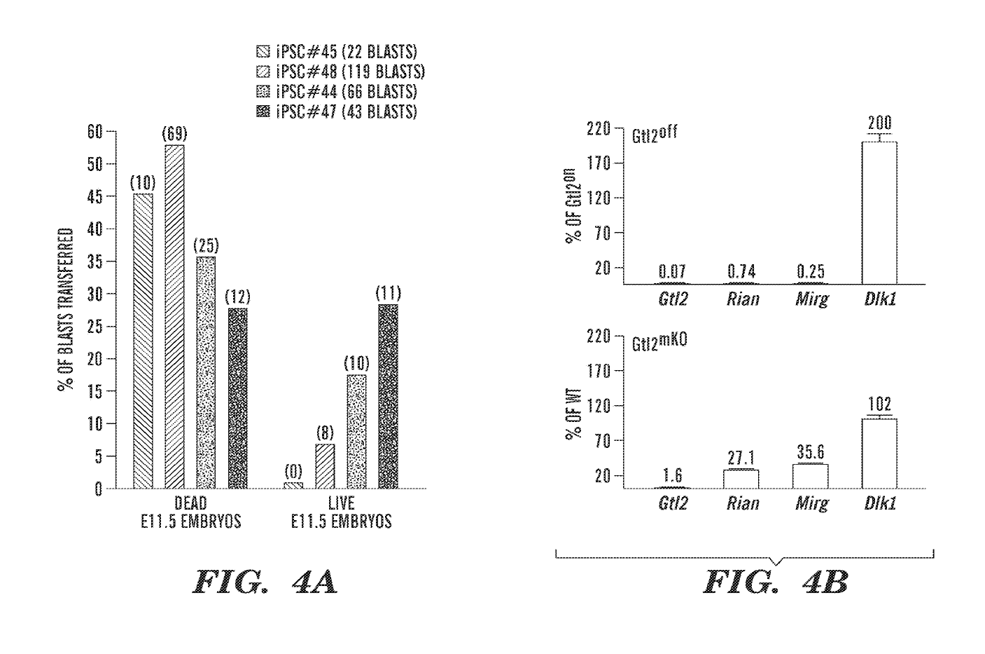

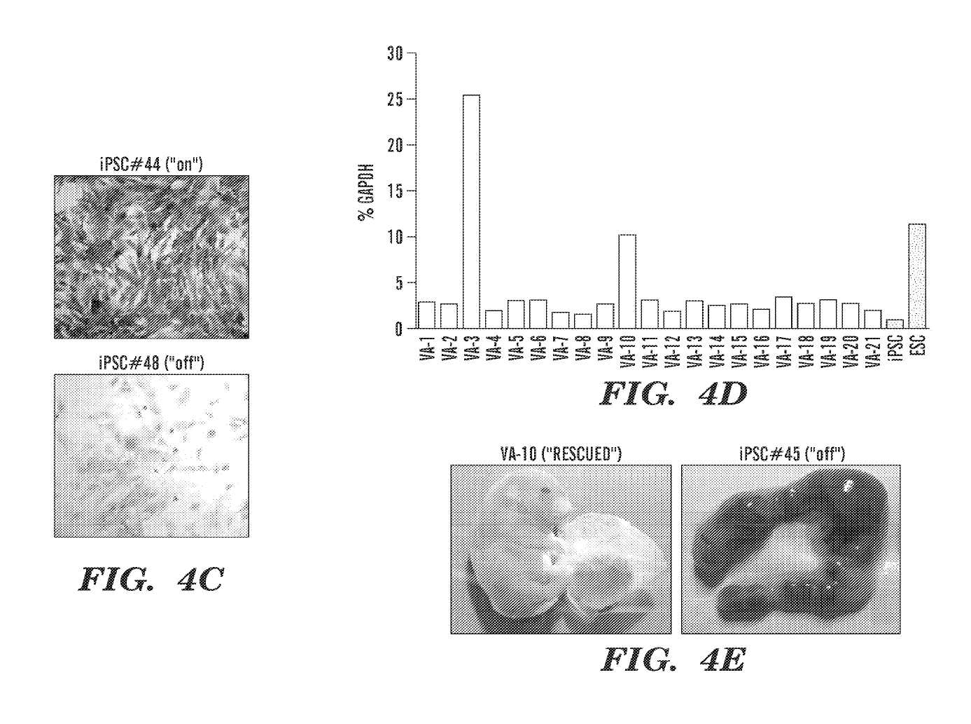

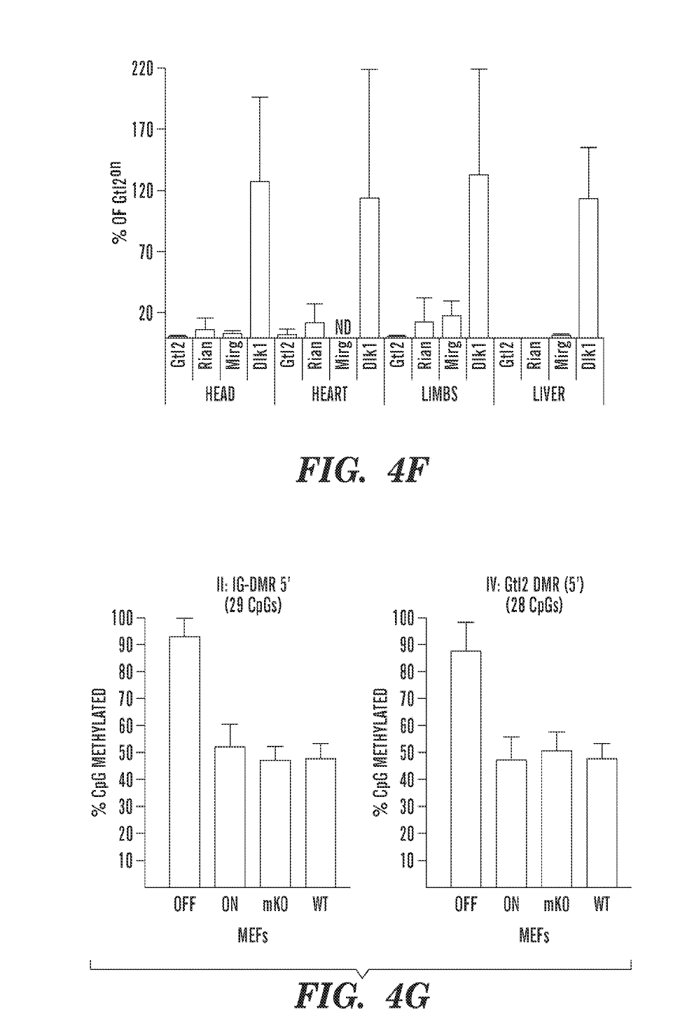

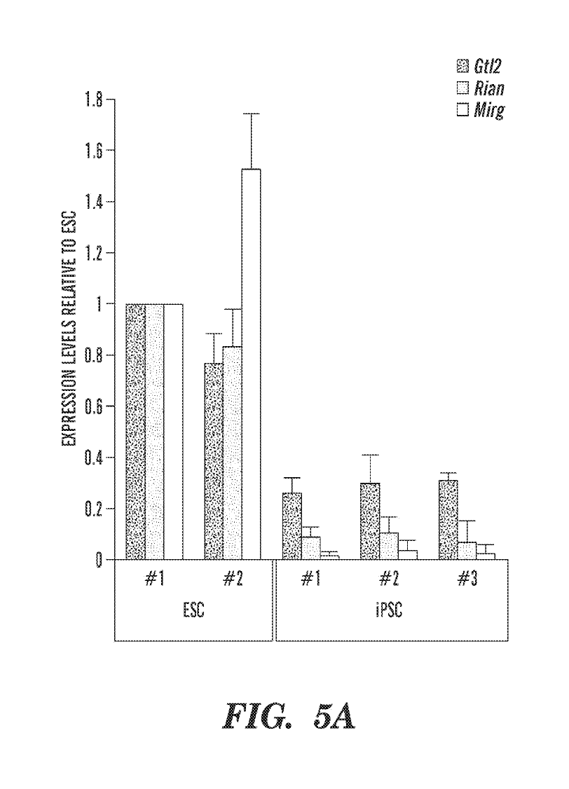

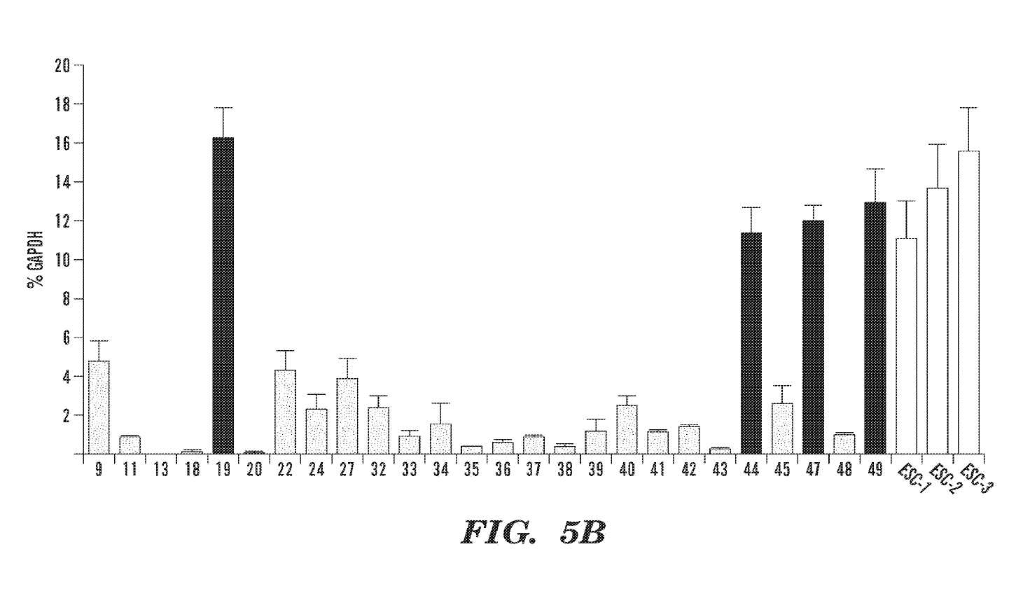

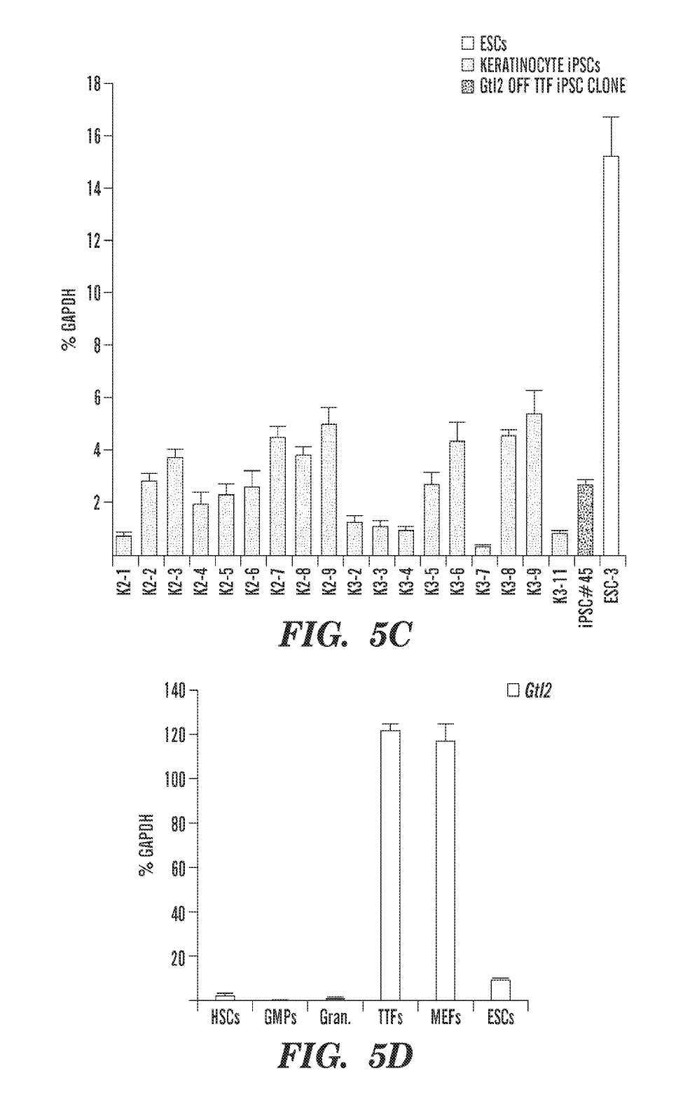

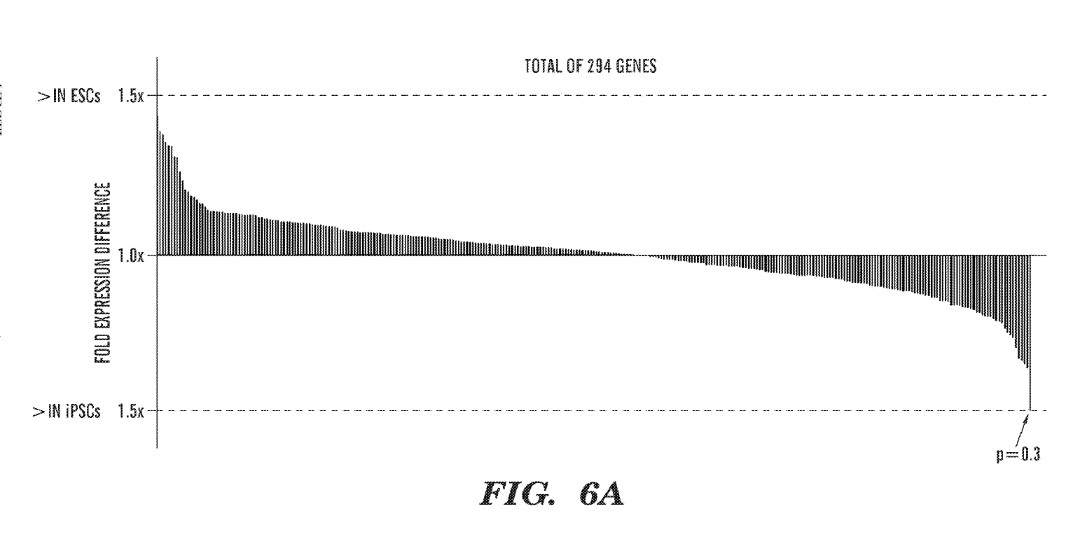

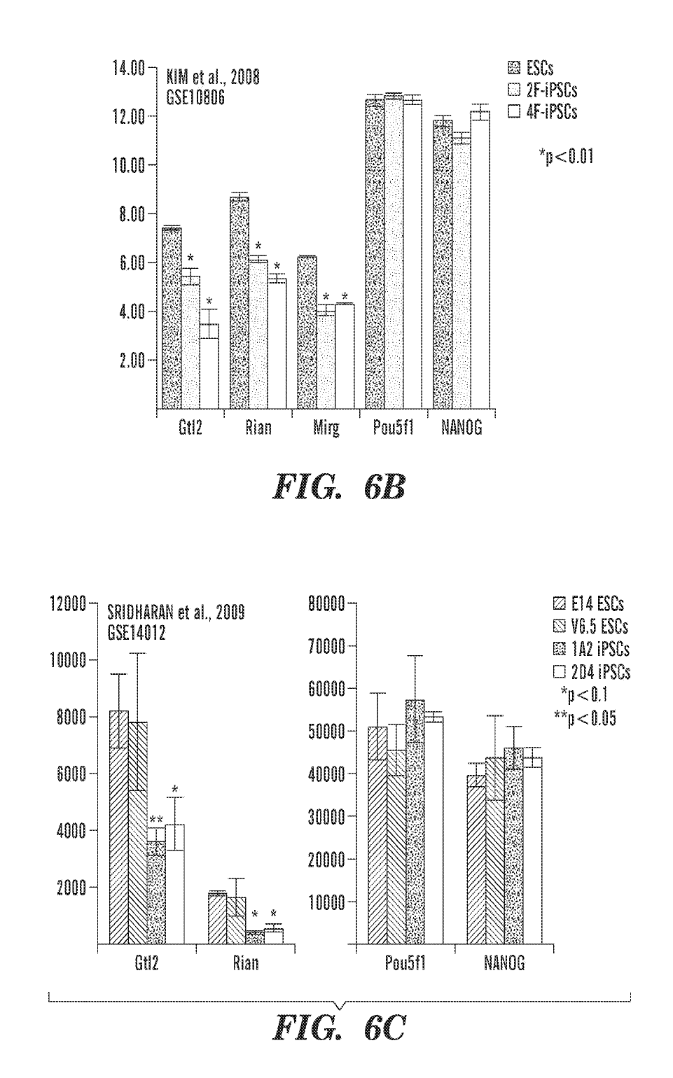

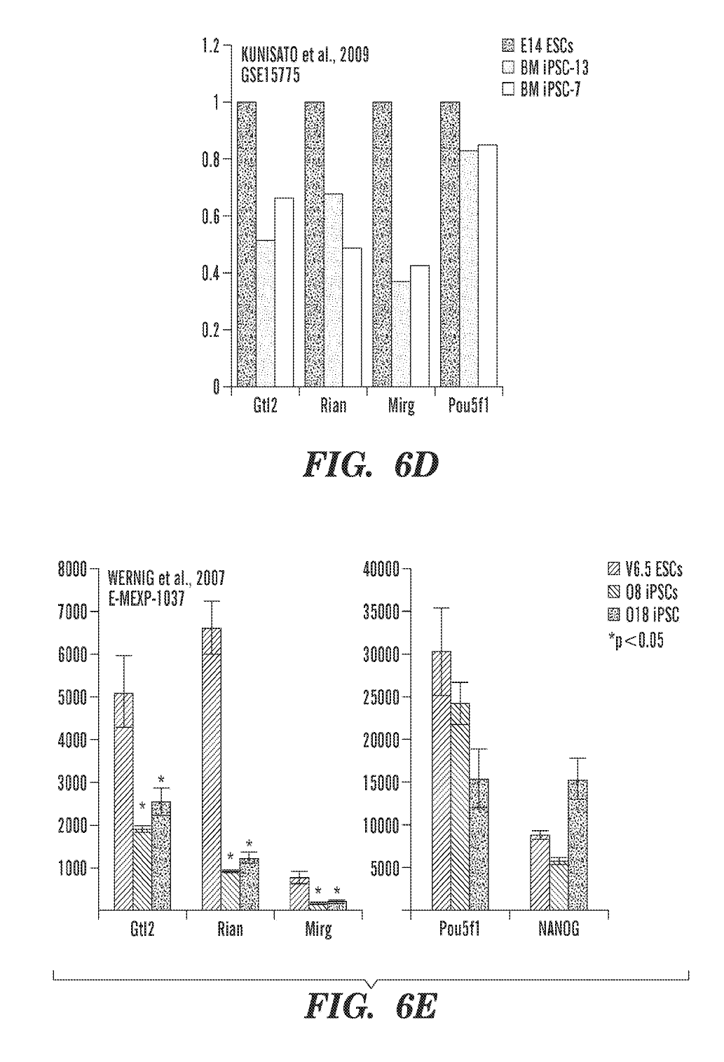

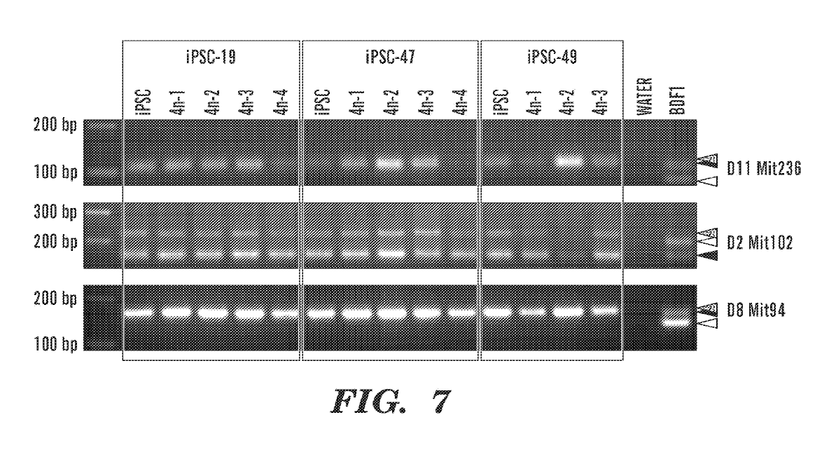

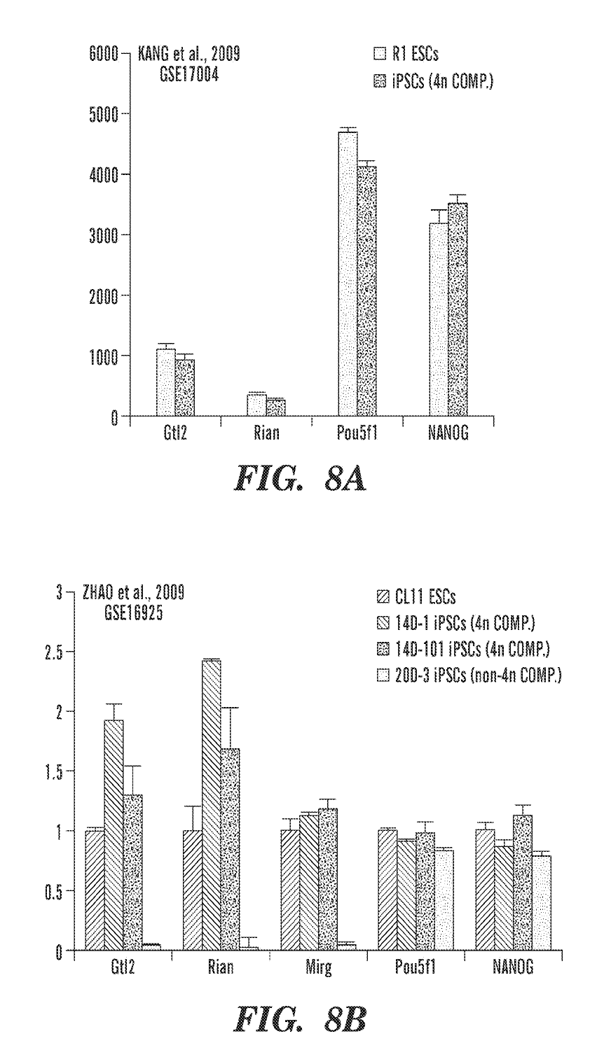

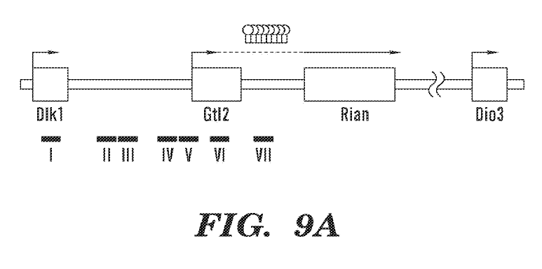

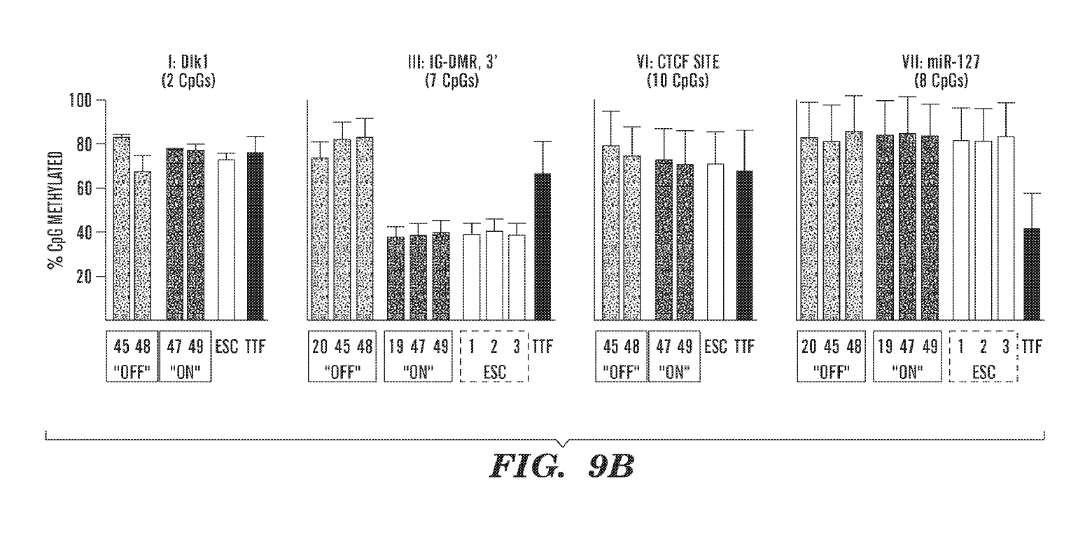

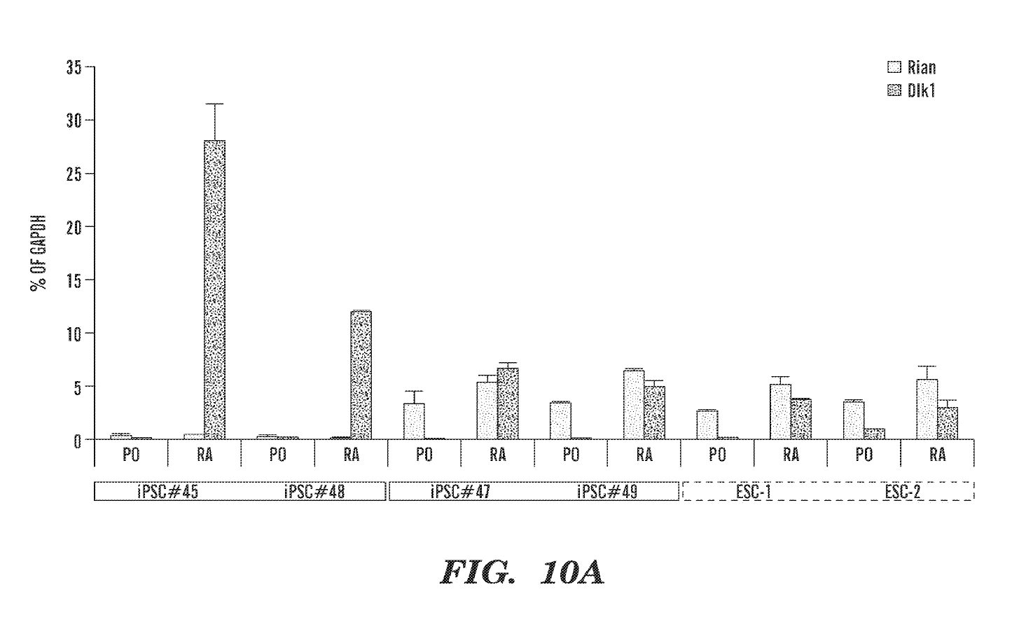

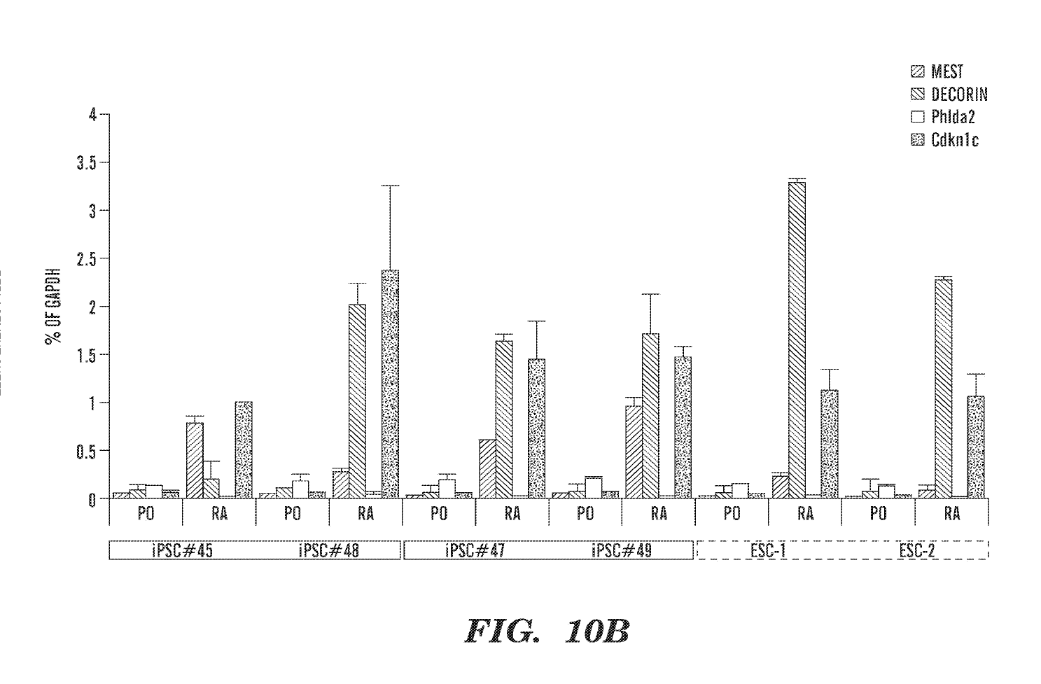

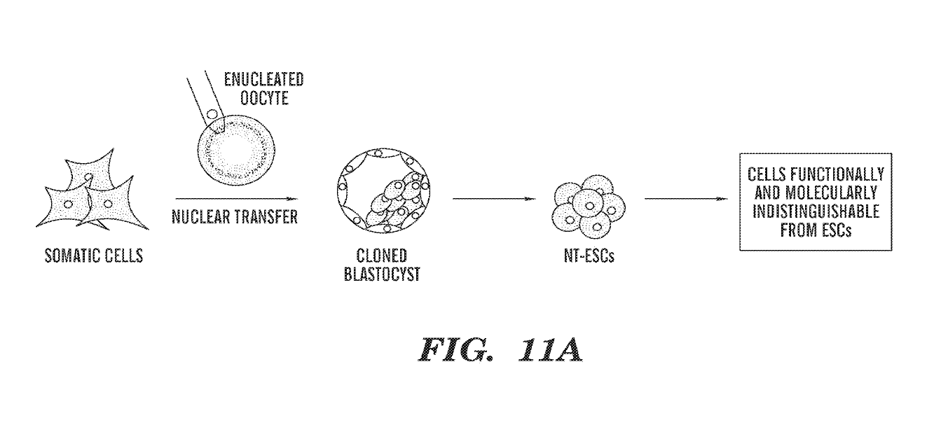

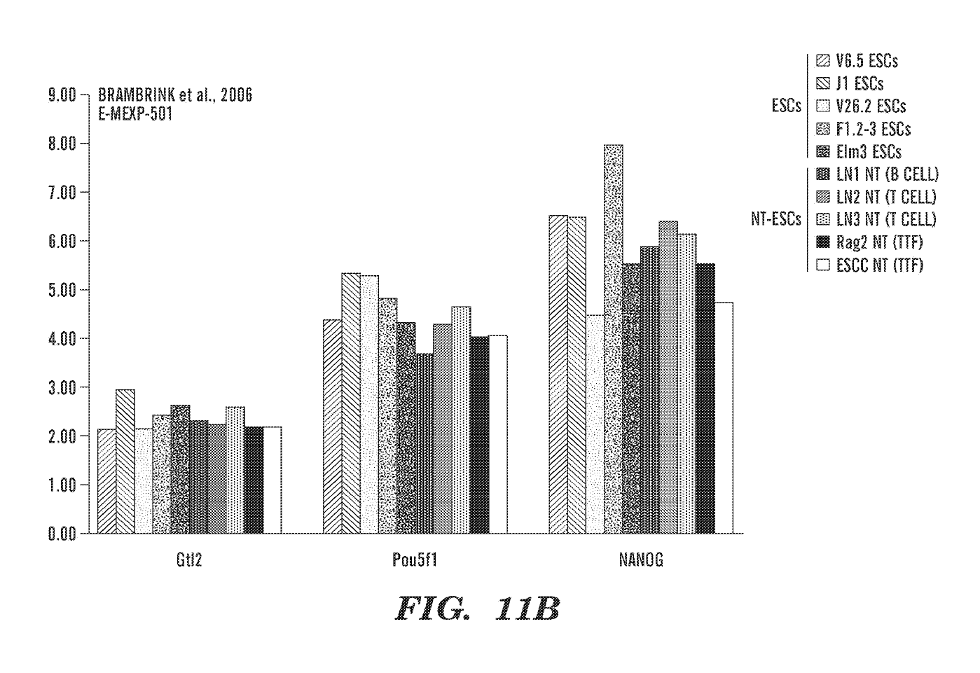

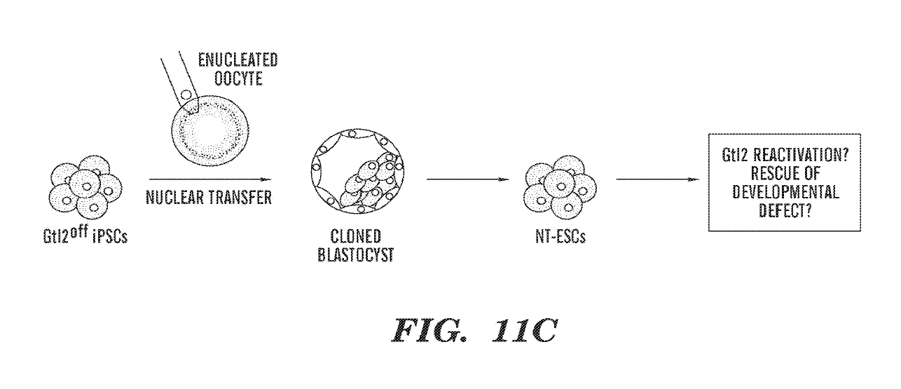

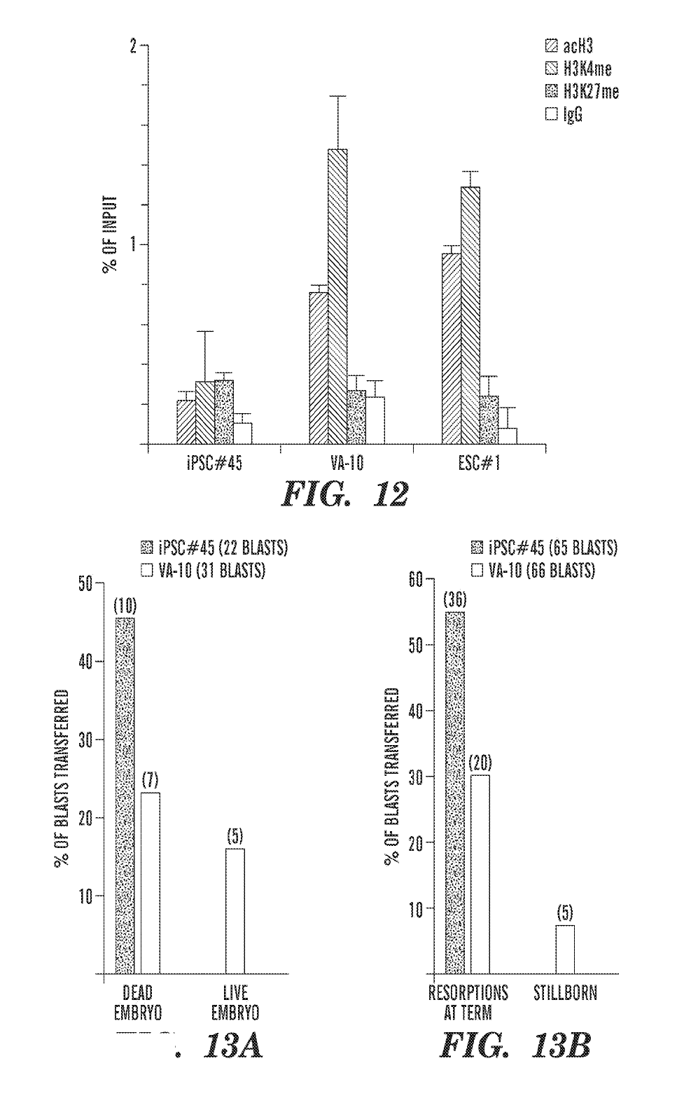

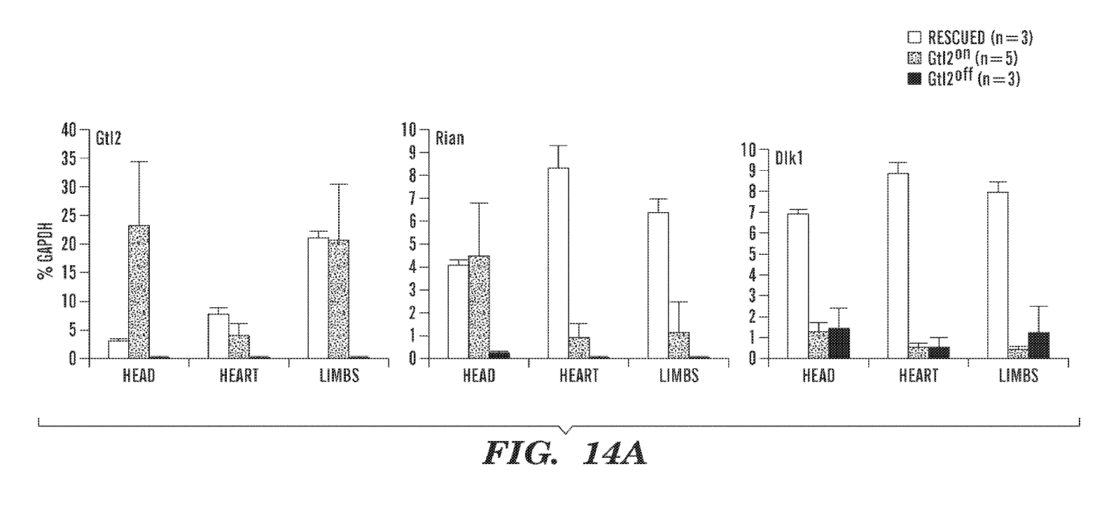

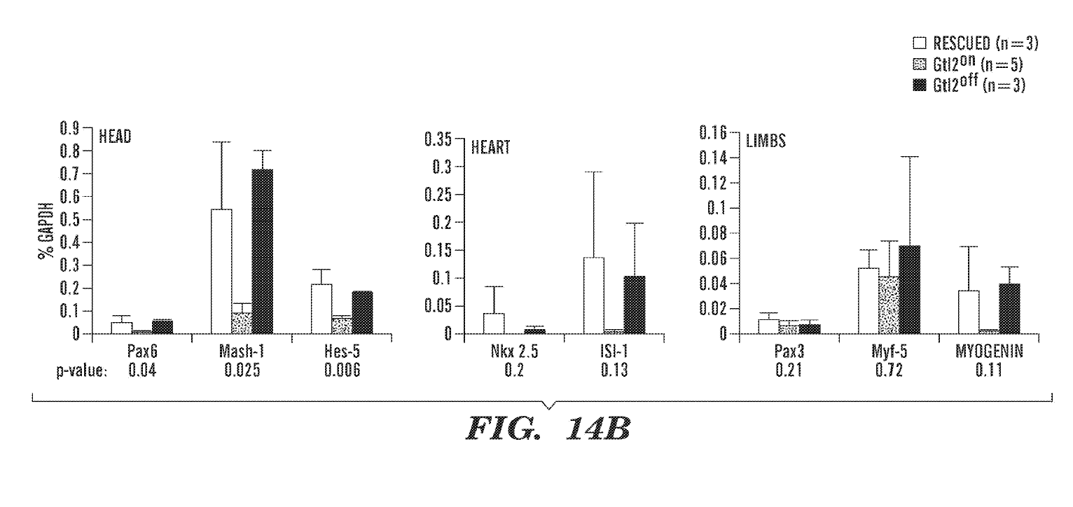

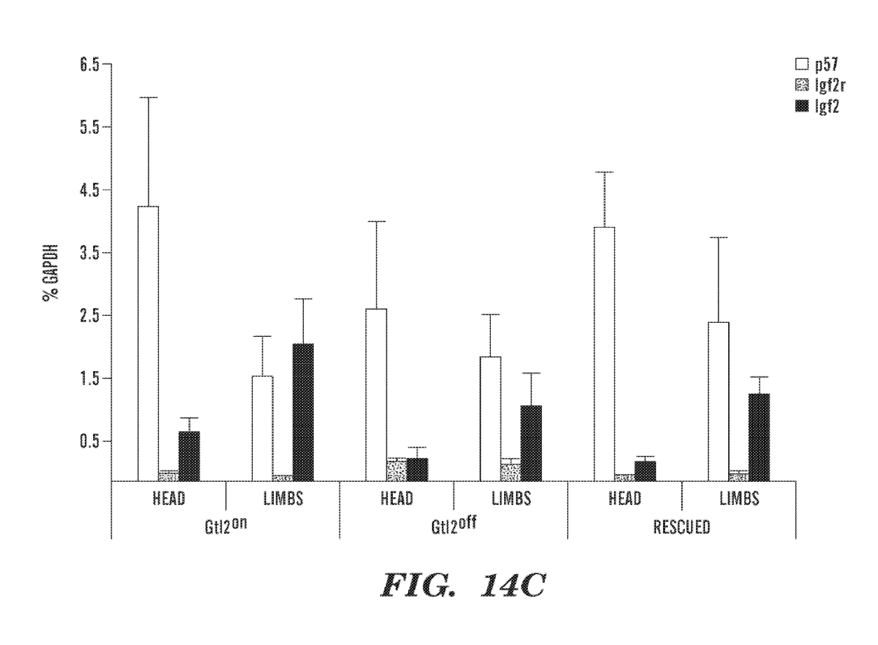

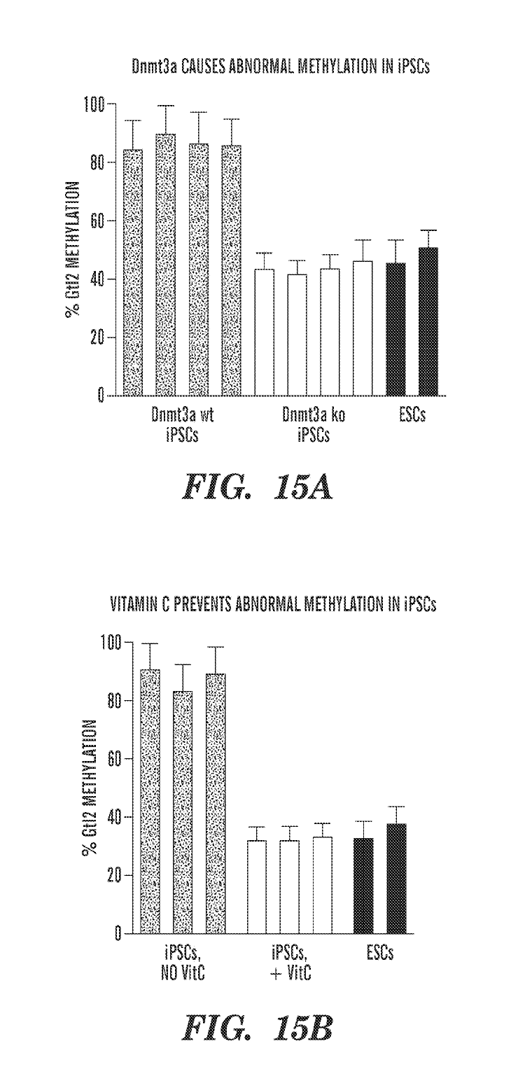

Genetically matched mouse ESCs and derivative iPSCs were used to screen for molecular and functional differences between these two pluripotent cell types. Briefly, a polycistronic cassette expressing Oct4, Klf4, Sox2, and c-Myc under the control of a doxycycline-inducible promoter was inserted into the Col1a1 locus of ESCs cells expressing the reverse tetracycline-dependent transactivator (rtTA) from the ROSA26 promoter (Stadtfeld, M. et al., Nature methods 7(1):53). These ESCs (designated Collagen-OKSM ESCs) were then used to generate mice from which different somatic cell types were isolated and induced with doxycycline to derive genetically matched iPSCs for molecular and functional comparisons (FIG. 1a,b).

First, the abilities of parental Collagen-OKSM ESCs and iPSCs derived from mouse embryonic fibroblasts (MEFs) that had been isolated from ESC-chimeric fetuses, to support the development of all-iPSC mice was compared using tetraploid (4n) embryo complementation (Nagy, A. et al., Development (Cambridge, England) 110(3):815 (1990); Eggan, K. et al., PNAS USA 98(11):6209 (2001)). In this assay, iPSCs or ESCs are injected into 4n host blastocysts, which can only give rise to extra-embryonic tissues, whereas the injected pluripotent cells generate the entire mouse conceptus. Two tested ESC lines gave rise to neonatal and adult mice at expected frequencies (13-20%)(Eggan, K. et al., PNAS USA 98(11):6209 (2001)), demonstrating that the OKSM transgene per se does not affect the developmental potential of these cells (Table 1). In contrast, all four tested iPSC lines repeatedly failed to support the development of all-iPSC mice, indicating qualitative differences between ESCs and these iPSC clones (Table 1).

It was reasoned that a transcriptional comparison of the iPSC lines, which failed in the 4n complementation assay, with their parental ESC lines that supported the development of all-ESC mice, might reveal molecular changes that could explain the developmental deficits of iPSCs. Global mRNA profiling showed striking similarities in the overall transcriptional patterns of four Collagen-OKSM ESCs and six iPSCs and did not separate these cells using unsupervised clustering or principal component analysis (FIG. 1c and data not shown). In fact, only two transcripts were identified as differentially expressed (>2-fold difference, t-test, p<0.05) between ESCs and iPSCs. These were the non-coding cDNA Gtl2 (also known as Meg3) and the small nucleolar RNA (snoRNA) Rian (FIG. 1d).

Meg3/Gtl2 and Rian localize to the imprinted Dlk1-Dio3 gene cluster on mouse chromosome 12qF1 and are maternally expressed in mammals (FIG. 1e) (da Rocha, S T et al., Trends Genet. 24(6):306 (2008)). Of note, both genes were strongly repressed in iPSC clones compared to ESC clones while expression of pluripotency and housekeeping genes remained unaffected (data not shown). Quantitative PCR (qPCR) analysis of Meg3/Gtl2, Rian and Mirg, another maternally expressed imprinted gene in the Dlk1-Dio3 cluster, confirmed transcriptional silencing in iPSCs (FIG. 5a).

Interestingly, expression of the paternally expressed Dlk1 gene, that also localizes to chromosome 12qF1, and of other imprinted genes including H19 and Igf2r, showed clone-to-clone variations, as was seen previously for ESCs (Humpherys, D. et al., Science 293 (5527):95 (2001)), but no consistent expression differences between ESCs and iPSCs. This shows that imprinted gene silencing is not a genome-wide phenomenon in iPSCs (Table 2). Moreover, none of the almost 300 genes that had previously been reported to be differentially expressed between iPSCs and ESCs (Chin, M H et al., Cell Stem cell 5(1):111 (2009)) was changed in Collagen-OKSM iPSCs (FIG. 6a). These data indicate that a relatively small set of transcripts distinguishes genetically matched iPSCs and ESCs and indicate that the majority of previously seen differences are likely due to variations in genetic background or viral transgene insertions.

Imprinting of the Dlk1-Dio3 locus is accompanied by differential expression of about 50 miRNAs that are also encoded within the gene cluster (FIG. 1e) (Seitz, H. et al., Nature genetics 34(3):261 (2003); Seitz, H. et al., Genome research 14(9):1741 (2004)). To evaluate if miRNAs are differentially expressed between ESCs and iPSCs, genome-wide miRNA profiling was performed on the same samples as analyzed for mRNA expression. Of 336 miRNAs detected, 21 (6.3%) were differentially expressed between all ESC and iPSC clones analyzed (data not shown and Table 3). Remarkably, all of these miRNAs localized to chromosome 12qF1 and were silenced in iPSC, thus corroborating the notion that most iPSCs show aberrant silencing of this major imprinting domain.

To determine the generality of Meg3/Gtl2 silencing in iPSCs, expression of Meg3/Gtl2 was analyzed in 61 additional iPSC lines derived from hematopoietic stem cells (HSC, 11 lines), granulocyte-macrophage progenitors (GMP, 11 lines), granulocytes (Gran, 9 lines), peritoneal fibroblasts (PF, 6 lines), tail tip fibroblasts (TTF, 6 lines) and keratinocytes (18 lines) (data not shown and FIG. 5b,c). Only four of these lines (5.8%), all originating from either peritoneal or tail tip fibroblasts, showed Meg3/Gtl2 expression levels similar to ESCs (termed “Gtl2^{on} clones”). The finding that the vast majority of iPSC clones derived from different somatic cell types showed partial or complete suppression of Meg3/Gtl2 expression (termed “Gtl2^{off} clones”) demonstrates that silencing of this locus occurs in iPSCs regardless of their cell of origin. In agreement with these data, analyses of published microarray datasets comparing ESCs and iPSCs derived from mouse fibroblasts, neural and bone marrow cells also showed repression of maternally expressed 12qF1 transcripts (FIG. 6b-e), supporting the notion that silencing of this cluster is common upon factor-mediated reprogramming. It is anticipated that similar expression abnormalities are seen in human iPSCs.

Dysregulation of genes within the Dlk1-Dio3 cluster can be detrimental during pre- and postnatal mouse development (Takahashi, N. et al., Human molecular genetics 18(10):1879 (2009); Lin, S P. et al., Development (Cambridge, England)134(2): 417 (2007); Steshina, E Y et al., BMC genetics 7: 44 (2006); Lin, S P et al., Nat Genet 35(1):97 (2003); da Rocha, S T et al., PLoS genetics 5(2):e1000392 (2009)). To assess whether the expression status of Meg3/Gtl2 and associated transcripts correlates with the developmental potential of iPSC, a total of nine Gtl2^{off} clones (3 HSC-iPSC, 1 GMP-iPSC, 2 PF-iPSC, 2 TTFiPSC) were injected into diploid blastocysts, which gave rise to 38 adult chimeras that exhibited low to medium degree (10-50%) coat color chimerism (FIG. 2a-c and Table 4). In contrast, three Gtl2^{on} iPSC clones (1 PF-iPSC, 2 TTF-iPSC) injected into diploid blastocysts yielded 11 adult mice with a coat color chimerism ranging from 70-100%, similar to the chimerism seen with ESCs (FIG. 2b and Table 4). Importantly, all four Gtl2^{on} iPSC clones supported the development of neonatal all-iPSC mice upon injection into 4n blastocysts at efficiencies similar to those seen with ESCs (7-19% for iPSCs compared with 13-20% for ESCs) (Table 1). It was confirmed that these mice were entirely iPSC-derived by PCR for strain-specific polymorphisms (FIG. 7), by detection of homogenous GFP fluorescence of all-iPSC neonates, originating from a ROSA26-EGFP allele that had been introduced into the parental ESCs, and by uniform agouti coat color of adolescent all-iPSC mice (FIG. 20. This is the first demonstration of animals produced entirely from adult-derived iPSCs. In contrast to Gtl2^{on} iPSC clones, injection of ten different Gtl2^{off} iPSC clones (4 MEF-iPSC, 1 HSC-iPSC, 1 GMP-iPSC, 1 PF-iPSC, 3 TTF-iPSC) into 4n blastocysts consistently failed to produce all-iPSC pups but instead resulted in resorptions (Table 1). Thus, the expression status of Meg3/Gtl2 in iPSCs predicts their developmental potential into chimeric and all-iPSC mice. It is anticipated that 4n-competent iPSC clones can be derived from somatic cells other than fibroblasts.

To test whether Gtl2^{on} and Gtl2^{off} iPSCs could be distinguished by the expression of other genes, global mRNA and miRNA expression profiling was performed for four fibroblast-derived non-4-n complementation-competent and four 4n complementation-competent iPSC lines. This analysis identified only Mega/Gtl2, Rian and a total of 26 miRNAs, which all localize to the Dlk1-Dio3 cluster, as differentially expressed (FIG. 2e and Table 5). The conclusion that the activation status of maternally expressed genes on chromosome 12qF1 is a strong indicator of the developmental potential of iPSCs was further supported by analysis of two published array datasets showing that Meg3/Gtl2 was expressed in ESCs and 4n complementation-competent iPSC lines but was downregulated in non-4-n complementation-competent iPSC lines (Zhao, X Y et al., Nature (2009); Kang, L. et al., Cell Stem Cell 5(2):135 (2009)) (FIG. 8).

Imprinting of the Dlk1-Dio3 cluster is regulated by differentially methylated regions (DMRs) that become epigenetically modified in the germline. These include an intergenic DMR (IG-DMR), located between the Dlk1 and Glt2 genes (da Rocha, S T et al., PLoS genetics 5(2):e1000392 (2009)), and a DMR spanning the Meg3/Gtl2 promoter (Gtl2 DMR)(Yu, J et al., Science 324(5928):797 (2009)). To determine whether aberrant DNA methylation might be responsible for the transcriptional silencing seen in Gtl2^{off} iPSC lines, the methylation status was compared for the IG-DMR and Meg3/Gtl2 DMR as well as that of three other CpG-rich regions on chromosome 12qF1 in ESCs, Gtl2^{on} iPSCs, Gtl2^{off} iPSCs and their parental tail-tip fibroblasts (FIG. 3a). As expected for germline imprinted regions, approximately 50% of CpGs within the IG-DMR and Meg3/Gtl2 DMR were methylated in fibroblasts, ESCs and Gtl2^{on} iPSCs, whereas close to 100% of CpGs within these DMRs were methylated in Gtl2^{off} iPSC lines (FIG. 3b and FIG. 8). The other CpG-rich regions analyzed remained unaffected (FIG. 9). Imprinting of the Dlk1-Dio3 cluster is also regulated by histone acetylation (Carr, M S. et al., Genomics 89(2):280 (2007)) and chromatin immunoprecipitation experiments indeed revealed a significant decrease in activation marks such as methylated H3K4 and acetylated H3 and H4 in Gtl2^{off} iPSC lines compared with Gtl2^{on} iPSC lines and ESCs (FIG. 3c). Without wishing to be bound by theory, these observations demonstrate that the normally expressed maternal Meg3/Gtl2 allele has acquired an aberrant paternal-like silenced state in Gtl2^{off} iPSC clones.

Imprinted gene expression is unstable in murine ESCs (Humpherys, D et al., Science 293 (5527): 95 (2001); Dean, W et al., Development (Cambridge, England) 125(12):2273 (1998)). To evaluate if silencing of the Dlk1-Dio3 locus in iPSCs is maintained, subclones from Gtl2^{off} and Gtl2^{on} iPSCs were derived and Mega/Gtl2 expression was assessed by qPCR. The Mega/Gtl2 locus remained silent in all Gtl2^{off} iPSC clones and continued to be expressed in all Gtl2^{on} iPSC clones, demonstrating stability of the Mega/Gtl2 expression state in undifferentiated iPSCs (FIG. 3d, top). This pattern was not altered if doxycycline was adminstered during the subcloning procedure (FIG. 3d, bottom), thus indicating that overexpression of the reprogramming factors in established iPSCs is insufficient to induce silencing.

To assess if silencing of Meg3/Gtl2 might be resolved during differentiation, Gtl2^{off} and Gtl2^{on} iPSCs as well as ESCs were exposed to the differentiation-stimulating agent retinoic acid (RA) for 5 days. Dramatic changes in cellular morphology and downregulation of Pou5f1 in all RA-treated clones indicated successful differentiation (FIG. 3e,f). Whereas Gtl2^{on} iPSCs and ESCs readily upregulated Meg3/Gtl2 (FIG. 3f, top) and Rian (FIG. 10) during differentiation, Gtl2^{off} iPSCs showed stable silencing of these genes, demonstrating that in vitro differentiation fails to reactivate maternally imprinted genes in the Dlk1-Dio3 cluster. The expression of imprinted genes outside of chromosome 12qF1 was not affected (FIG. 3f, bottom, and FIG. 10)

Because Gtl2^{off} iPSC clones failed to produce viable all-iPSC mice, it was next sought to determine if they could autonomously support development into early embryos. Injection of Gtl2^{off} and Gtl2^{on} iPSC clones into 4n blastocysts gave rise to normal-appearing embryos at midgestation (E1 1.5) (data not shown). However, the number of living E1 1.5 embryos obtained from Gtl2^{off} iPSC clones was reduced compared with embryos obtained from Gtl2^{on} iPSC clones (FIG. 4a), indicating that Gtl2^{off} mice die around this developmental stage. This phenotype resembles that of mice with paternal uniparental disomy of distal chromosome 12 (Tevendale, M. et al., Cytogenet Genome Res 113 (1-4): 215 (2006)), which die before E16.5, but is distinct from that of maternal Gtl2 knock-out mice)(Gtl2^{mKO}), which die perinatally (Takahashi, N. et al., Human molecular genetics 18(10):1879 (2009)). The less severe phenotype of Gtl2^{mKO} embryos compared with Gtl2^{off} embryos might be due to the comparably modest reduction in maternally expressed 12qF1 genes seen in Gtl2^{mKO} mice (Takahashi, N. et al., Human molecular genetics 18(10):1879 (2009)). For example, Rian and Mirg transcripts were low but detectable in Gtl2^{mKO} MEFs (FIG. 4b). In contrast, these genes were almost completely silenced in MEFs and different tissues derived from Gtl2^{off} all-iPSC embryos (FIG. 4c,d). Notably, expression of the Dlk1 gene, which is reciprocally imprinted to Meg3/Gtl2 (Schmidt, J V et al., Genes & development 14(16):1997 (2000)), was upregulated in Gtl2^{off} MEFs but not in Gtl2^{mKO} MEFs (FIG. 4b), further supporting the observation that the maternal Dlk1-Dio3 cluster has acquired a paternal-like expression state. Accordingly, the IG-DMR and Gtl2-DMR were hypermethylated in Gtl2^{off} MEFs but remained unaffected in Gtl2^{mKO} MEFs (FIG. 4e). Together, these observations are in agreement with the notion that stable transcriptional repression of the Dlk1-Dio3 locus is the cause for the developmental failure of Gtl2^{off} all-iPSC embryos.

ESCs derived from cloned embryos are transcriptionally identical with ESCs produced from fertilized embryos and also support the development of all-ESC mice, regardless of donor cell identity (Brambrink, T. et al., PNAS USA 103(4):933 (2006)), indicating that nuclear transfer (NT) generates faithfully reprogrammed pluripotent cells (Supplementary FIG. 7a). In agreement with this observation, Meg3/Gtl2 is expressed in 4n complementation-competent control ESC and NT ESC lines derived from fibroblasts and hematopoietic cells (FIG. 11b). It was tested whether NT could reverse the aberrant silencing of genes within the Dlk1-Dio3 cluster in Gtl2^{off} iPSCs and rescue their ability to support the development of all-iPSC mice (FIG. 11c). To this end, nine NT ESC lines from Gtl2^{off} iPSCs were derived from TTFs and fetal liver cultures using adenoviral vectors (Stadtfeld, M. et al., Science 322 (5903):945 (2008)) or from hematopoietic stem cells and granulocytes using the Collagen-OKSM system. Some of these iPSCs were germline competent (Stadtfeld, M. et al., Science 322 (5903):945 (2008)), indicating that they were genetically normal, but failed to give rise to all-iPSC mice (Table 6). Global transcriptome analysis showed no consistent differences in mRNA and miRNA expression profiles between NT ESCs and the donor iPSC clones. Most importantly, Meg3/Gtl2 and Rian remained repressed in all NT iPSCs (FIG. 11d). Accordingly, these cells failed to generate all-iPSC mice (Table 6), indicating that NT cannot reset the aberrant gene expression patterns and rescue the limited developmental potential acquired during iPSC generation. This notion is consistent with the previous finding that aberrant genomic imprints present in somatic donor cells cannot be restored in cloned animals following nuclear transfer (Humpherys, D. et al., Science 293(5527):95 (2001)).

Given that Gtl2^{off} iPSC clones showed reduced histone acetylation at the Meg3/Gtl2 locus (FIG. 3c), it was postulated whether treatment of Gtl2^{off} iPSC clones with the histone deacetylase inhibitor valproic acid (VA) could reactivate the silenced gene cluster. Indeed, two out of 21 subclones treated with VA exhibited increased Meg3/Gtl2 expression with one iPSC clone showing expression levels comparable to ESCs (FIG. 4f). Consistent with transcriptional reactivation of the cluster, re-appearance of H3K4 methylation and H3 acetylation was observed at the Meg3/Gtl2 locus in this rescued clone (FIG. 12). Injection of this clone into 4n blastocysts gave rise to apparently normal midgestation (E1 1.5) embryos at frequencies similar to those seen with Gtl2^{on} iPSC clones (FIG. 4b and FIG. 13a). These embryos expressed Meg3/Gtl2, Rian and Mirg at significantly higher levels compared with embryos produced with Gtl2^{off} iPSC clones (FIG. 14a) and also showed normal expression levels of tissue-specific marker genes such as Mash-1 and Hes-5 that were repressed in Gtl2^{off} embryos and thus may represent direct or indirect targets of one of the miRNAs encoded in Dlk1-Dio3 (FIG. 14b). Importantly, the rescued clone supported the development of several full-term pups, which was not seen with the parental iPSCs or any other Gtl2^{off} clone (FIG. 4g and FIG. 139b). These pups were severely overgrown, however, and hence non-viable.

Without wishing to be bound by theory, it was surmised that the observed overexpression of Dlk1 in the rescued iPSC clone (FIG. 14a), which causes neonatal lethality due to fetal overgrowth (da Rocha, S T et al., PLoS genetics 5(2):e1000392 (2009)), is responsible for this phenotype. Alternatively, VA treatment may have caused the dysregulation of other genes even though no aberrant expression of several candidate imprinted genes implicated in growth control (FIG. 14c) was observed.

These data show that the expression of a surprisingly small number of transcripts and miRNAs, which localize to a single cluster in the genome, distinguishes mouse iPSCs from ESCs and is predictive for their developmental potential. It is anticipated that human iPSCs show a similar dysregulation of genes, which will affect their utility in drug screening and therapy. Understanding the causes for the specific silencing of the Dlk1-Dio3 cluster during factor-mediated reprogramming will shed light on the molecular mechanisms of reprogramming as well as on the epigenetic regulation of this particular locus.

### Example 2

### Exemplary Methods for Use with the Methods Described Herein ESC and iPSC Derivation

Collagen-OKSM ESCs were generated by introducing a doxycycline-inducible version of a polycistronic reprogramming cassette encoding for Oct4, Klf4, Sox2 and c-Myc into the Collagen 1A1 locus. ESCs were then injected into blastocysts to derive chimeric mice, which were bred with ROSA26-M2-rtTA mice to derive a reprogrammable mouse strain (Stadtfeld, M. et al., Nature methods 7(1):53). Somatic cells were isolated from ESC-chimeras or the reprogrammable mouse strain and cultured in the presence of doxycycline to obtain iPSCs. ESCs and iPSCs were cultured under standard mouse ESC conditions.

### Gene Expression Analyses

Total RNA was isolated from ESCs and iPSCs after removal of feeder cells and subjected to transcriptomal analyses using either Affymetrix U-133 μlus2.0 mRNA expression arrays (for mRNA analysis) or the miRCURY™ LNA Array (Exiqon) (for miRNA analysis).

### Epigenetic Analyses

Genomic DNA was isolated from purified cell populations, bisulfite-converted and analyzed by pyrosequencing. For analysis of histone modifications, chromatin immunoprecipitation was performed with antibodies against anti-aCH3 (06-5 99 Millipore), anti-aCH4 (06-866,Millipore), anti-dimethyl K4 of H3 (07-030, Millipore), anti-trimethyl K27 of H3 (ab6002, Abcam).

### Generation of OKSM ESCs

A polycistronic cassette encoding Oct4, Klf4, Sox2 and c-Myc was cloned into the shuttle plasmid pBS31 using NotI/ClaI digestion. The resulting plasmid was electroporated into KH2 ESCs (Beard, C. et al., Genesis 44(1):23 (2006)) together with a plasmid driving expression of Flp recombinase. Correctly targeted clones were isolated by hygromycin selection and confirmed by Southern blot analysis as previously described (Beard, C. et al., Genesis 44(1):23 (2006)). Individual OKSM ESC subclones were gene targeted with ROSA26-EGFP as has been described previously (Hochedlinger, K. et al., Cell 121(3):465 (2005)) to facilitate tracking of ESC-derived cells after blastocyst injection. OKSM ESCs and derivative mice are described in detail elsewhere (Stadtfeld, M et al., Nature methods 7(1):53).

### Cell Culture

ESCs and iPSCs were cultured in ESC medium (DMEM with 15% FBS, L-Glutamin, penicillin-streptomycin, non-essential amino acids, 3-mercaptoethanol and 1000 U/ml LIF) on irradiated feeder cells. Mouse embryonic fibroblasts (MEFs) were isolated by trypsin-digestion of midgestation (E14.5) ESC-chimeric embryos followed by culture in fibroblast medium (DMEM with 10% FBS, L-Glutamin, penicillin-streptomycin, nonessential amino acids and 3-mercaptoethanol). 2 μg/ml puromycin was added to these cultures for five days to selected for ESC-derived cells. Tail-tip fibroblast (TTF) cultures were established by trypsin digestion of tail-tip biopsies taken from newborn (3-8 days of age) chimeric mice derived after blastocyst injection of ROSA26-EGFP targeted ESCs. ESC-derived cells were isolated based on GFP expression and maintained in fibroblast medium. For the establishment of peritoneal fibroblast (PF) cultures, adult OKSM strain mice were euthanized and roughly 1 square centimeter of peritoneal muscle isolated and chopped into small pieces in 0.25% Trypsin/EDTA in a 35 mm cell culture vessel. After five minutes of incubation at 37° C., 6 ml fibroblast medium was added and the tissue resuspended several times through a pipette. PF cultures were maintained and propagated like MEF and TTF cultures. Hematopoietic cells were isolated from peripheral blood and bone marrow as previously described (Eminli, S et al., Nature genetics 41(9):968 (2009)). Briefly, freshly isolated bone marrow cells were isolated by FACS using the following surface marker combinations: CD150⁺CD48⁻ckit⁺Sca-1iineage⁻ for HSCs, FcyR⁺CD34⁺ckit⁺Sca-1iineage⁻ for GMPs and CD11b^{high}Gr-1^{high}ckit⁻ for granulocytes. Sorted cells were immediately plated on top of irradiated feeder layers in ESC medium containing doxycycline. For HSCs and GMPs, the medium was supplemented with Flt3-ligand (10 ng μl⁻¹), SCF (10 ng μl⁻¹) and TPO (10 ng μl⁻¹). Doxycycline was withdrawn from all cells after two weeks and colonies picked and expanded using standard ESC culture techniques.

Reprogramming into iPSCs

Collagen-OKSM MEFs, TTFs and PFs were counted and seeded in fibroblast media at the desired density onto gelatin-coated plates that contained a layer of irradiated feeder cells. The next day, ES medium containing 2 μg/ml doxycycline was added and replenished every 3 days. Upon doxycycline withdrawal, cultures were washed twice with PBS and then continued in standard ESC medium until colonies were picked.

### RNA Isolation

ESCs and iPSCs grown on 35 mm dishes were harvested when they reached about 50% confluency and pre-plated on non-gelatinized T25 flasks for 45 minutes to remove feeder cells. Cells were spun down and the pellet used for isolation of total RNA using the miRNeasy Mini Kit (QIAGEN) without DNase digestion. RNA was eluted from the columns using 50 μl RNase-free water or TE buffer, pH7.5 (10 mM Tris-HCl and 0.1 mM EDTA) and quantified using a Nanodrop (Nanodrop Technologies).

### Quantitative PCR

cDNA was produced with the First Strand cDNA Synthesis Kit (Roche) using 1 μg of total RNA input. Real-time quantitative PCR reactions were set up in triplicate using 5 μl of cDNA (1:100 dilution) with the Brilliant II SYBR Green QPCR Master Mix (Stratagene) and run on a Mx3000P QPCR System (Stratagene). Primer sequences are listed in Table 6.

mRNA Profiling

Total RNA samples (RIN >9) were subjected to transcriptomal analyses using Affymetrix U-133plus2.0 mRNA expression microarray as previously described (Coser, K R. et al., PNAS USA 100(24):13994 (2003)). Hierarchical clustering was performed using Cluster and Treeview software (Eisen, M B et al., PNAS USA 95(25): 14863 (1998)) as well as the GeneSifter server (Geospiza, Seattle).

miRNA Profiling

Total RNA was subjected to quality control consisting of RNA measurement on the Nanodrop (OD260/230 and OD260/280 had to be greater than 1.8) and a run on the Agilent Bioanalyser 2100 (RIN values had to be higher than 7). The samples were then labeled using the miRCURY™ Hy3™/Hy5™ power labeling kit (Exiqon) and hybridized on the miRCURY™ LNA Array (v.11.0) (Exiqon). Labeling was determined to be successful when all capture probes for the control spike in oligo nucleotides produced signals in the expected range. The quantified signals were normalized using the global Lowess (LOcally WEighted Scatterplot Smoothing) regression algorithm.

### Blastocyst Injections

2n and 4n blastocyst injections were performed as described before (Eggan, K et al., PNAS USA 98(11):6209 (2001)). Briefly, female BDF1 mice were super-ovulated by intraperitoneal injection of PMS and hCG and mated to BDF1 stud males. Zygotes were isolated from females with a vaginal plug 24 hour after hCG injection. Zygotes for 2n injections were in vitro cultured for 3 days in vitro in KSOM media, blastocysts were identified, injected with ESCs or iPSCs and transferred into pseudopregnant recipient females. For 4n injections, zygotes were cultured overnight until they reached the 2-cell stage, at which point they were electrofused. One hour later, 1-cell embryos were carefully identified and separated from embryos that had failed to fuse, cultured in KSOM for another 2 days and then injected.

### Nuclear Transfer

Nuclear transfer was performed as previously described (Ono. Y and T. Kono, Biology of reproduction 75(2):210 (2006)). Briefly, donor iPSCs were cultured in collagen-coated dishes without a feeder layer for 3 days in standard ESC medium. To synchronize cells at metaphase, the cultures were cultured for 2 h in a medium containing 0.4 μg/mlnocodazole (Sigma-Aldrich), a microtubule polymerization inhibitor. Cells floating in the medium were collected. While being sucked into a transfer pipette, only the cells arrested at metaphase were selected and used as nuclear donors. The recipient oocytes were collected from mature B6CBF1 female mice. Micromanipulations were performed in M2 medium containing 5 μg/ml cytochalasin B (Sigma) and 1 μg/ml nocodazole in a micromanipulation chamber. Explanation of cloned blastocysts and ESC-derivation was done as described previously (Ono. Y and T. Kono, Biology of reproduction 75(2):210 (2006)).

### Chromatin Immunoprecipitation

20 million iPSCs, ESCs or MEFs were fixed with 1% formaldehyde for 10 minutes at room temperature (RT) and then lysed in 1 ml lysis buffer (50 mM Tris-HCl, pH 8.0, 10 mM EDTA, 1% SDS, protease inhibitors) for 20 minutes on ice. The lysate was split into three tubes and sonicated using Bioruptor for five times five minutes at high intensity, 30 sec on-30 sec off. After 10 minutes centrifugation, the supernatant was precleared for 1 hour at 4° C. with agarose beads preblocked with BSA (1 mg BSA for 10 ml beads) in IP Buffer (50 mMM Tris-HCl, pH8, 150 mM NaCl, 2 mM EDTA, 1% NP-40, 0.5% Sodium Deoxycholate, 0.1% SDS, protease inhibitors). 100 ml of precleared chromatin per reaction diluted in 1 ml IP Buffer in presence of 2 ug antibody were used for each immunoprecipitation reaction according to manufacturer's protocol. The antibodies used for this study were: anti-aCH3 (06-5 99 Millipore), anti-acH4 (06-866, Millipore), anti-dimethyl K4 of H3 (07-030, Millipore), anti-trimethyl K27 of H3 (ab6002, Abcam) and normal rabbit IgG (Millipore). The precipitate was purified using Qiaquick PCR purification kit and was analyzed by qPCR using Brilliant II SYBR Green qPCR Master Mix (600828, Agilent Technologies) using the sequence specific primer sets. Gtl2: 5′-AGCCCCTGACTGATGTTCTG-3′ (FWD) and 5′-TGGAAGGGCGATTGGTAGAC3′ (REV) and Pou5f1: 5′-GGAGGTGCAATGGCTGTCTTGTCC-3′ (FWD) and 5′-CTGCCTTGGGTCACCTTACACCTCAC-3′ (REV).

### In Situ Hybridization

MEFs grown on coverslips were fixed with 4% formaldehyde/5% acetic acid in PBS for 15 minutes at RT. After extensive PBS washes, they were dehydrated in 70% ethanol and left overnight at 4° C. The next day, they were rehydrated in a series of ethanol dilutions and incubated in hybridization buffer (50% formamide-5×SSC-RNase inhibitors) for 1 hour at 65° C. The hybridization was done overnight in a humidified chamber using 400 ng of sense or anti-sense Gtl2 specific probe/ml of hybridization buffer. The sense and antisense probes were synthesized by in vitro transcription with DIG RNA labeling mix (Roche) and SP6 and T7 polymerase, respectively, using Gtl2 cDNA amplified with the primers 5′-CTCTCGGGACTCCTGGCTCCAC-3′ (FWD) and 5′-GGGTCCAGCATGTCCCACAGGA-3′ (REV). The cells were serially washed and stained with an anti-DIG AP conjugated FAB fragment (1:2000 in blocking buffer) for 1 hour at RT. The detection was performed with NBT/BC1P reagent.

### Pyrosequencing

Genomic DNA was isolated using the DNeasy Blood & Tissue Kit (QIAGEN). ESCs and iPSCs were preplated onto cell culture vessels for 45 minutes after harvesting to remove feeder cells. Genomic DNA was bisulfate-converted using the EpiTect Bisulfite Kit (QIAGEN) with 400 ng of input DNA. DNA was eluted with 10 ml and 1 ml of it was used for PCR. PCR products were sequenced using the Pyrosequencing PSQ96 HS System (Biotage AB) following the manufacturer's instructions. The methylation status of each locus was analyzed using QCpG software (Biotage).

### Example 3

### Induced Increase in the Frequency of “Normal iPSCs”

We recently found that most iPSC lines have a more limited developmental potential than embryonic stem cells (ESCs), due to the aberrant silencing of important regulatory genes on chromosome 12 (called Dlk1-Dio3 cluster) in these iPSCs. A screen for molecules that might ameliorate this reprogramming abnormality was performed and two approaches were found that dramatically increased the frequency of “normal iPSCs”. These are 1) repression of an enzyme called Dnmt3a or 2) addition of ascorbic acid (Vitamin C) to do cell culture media during reprogramming (FIG. 15). This suggests that these straightforward modifications of the reprogramming procedure can be used to reproducible generate high-quality iPSCs at high frequency that can be used for disease modeling the study of developmental processes.

Reprogramming of Dnmt3a-Deficient Fibroblasts

Mouse embryonic fibroblasts (MEFs) harboring two conditional (“foxed”) alleles of Dnmt3a (Kaneda, M. et al. Essential role for de nova DNA methyltransferase Dnmt3a in paternal and maternal imprinting. Nature 429(6994):900-3) were co-transduced with a retrovirus encoding Cre recombinase (to inactivate Dnmt3a) and a lentivirus encoding a polycistronic reprogramming cassette. Emerging iPSC clones were picked two weeks later and propagated under standard ESC culture conditions. Excision of Dnmt3a was confirmed by polymerase chain reaction (PCR) specific for the inactivated allele.

### Derivation of iPS Cells in the Presence of Ascorbic Acid

MEFs with a doxycycline-inducible polycistronic reprogramming cassette in their genome (Stadtfeld, M. et al. A reprogrammable mouse strain from gene-targeted embryonic stem cells. Nature methods 7(1):53-5) were reprogrammed into iPS cells by culture in either doxycycline-containing ESC media or doxycycline-containing ESC media with 50 ng/μl ascorbic acid (Sigma A4544). Doxycline was withdrawn after 10-14 days of culture and emerging iPS cell colonies picked and expanded in regular ESC culture media (without doxycycline, without ascorbic acid). Pluripotency of established iPS cell clones was confirmed by marker gene expression and blastocyst injections.

### Analysis of DNA Methylation at the Dlk1-Dio3 Locus

Genomic DNA was isolated using the DNeasy Blood and Tissue Kit (Qiagen) and bisulfite converted using the EpiTect Bisulfite Kit (Qiagen) with 100-1000 ng input. Converted DNA was used for PCR to amplify the regulatory IG-DMR region and PCR products were sequenced using the Pyrosequencing PSQ96 HS System (Biotage AB) (Stadtfeld, M. et al. Aberrant silencing of imprinted genes on chromosome 12qF1 in mouse induced pluripotent stem cells).

1. A method for selecting an induced pluripotent stem cell (iPS), the method comprising: selecting an iPS cell that expresses a gene in the Dlk1-Dio3 cluster from a population of iPS cells.

2. The method of claim 1, wherein the gene is Meg3, Rian or Mirg.

3. The method of claim 1, wherein expression of each of genes Meg3, Rian and Mirg are measured.

4. The method of claim 1, wherein the induced pluripotent stem cell is a mammalian iPS cell.

5. The method of claim 1, further comprising differentiating the iPS cell selected in claim 1.

6. The method of claim 1, wherein the iPS cell expressing the identified gene in the Dlk1-Dio3 cluster has an enhanced differentiation potential compared to an iPS cell lacking expression of the identified gene in the Dlk1-Dio3 cluster.

7-19. (canceled)

20. A method for screening for an agent that enhances iPS cell differentiation potential, the method comprising:
(a) providing an iPS cell population lacking expression of one or more genes in the Dlk1-Dio3 cluster,
(b) contacting the iPS cell population with a candidate agent;
(c) measuring the level of expression of the one or more genes in the Dlk1-Dio3 cluster, wherein expression of the one or more genes is indicative that the agent enhances iPS cell differentiation potential.

21. The method of claim 20, wherein the one or more genes is Meg3, Rian or Mirg.

22. (canceled)

23. The method of claim 20, wherein the iPS cell in step (a) is genetically matched to the embryonic stem cell.

24. The method of claim 20, further comprising a step of comparing the epigenetic status of the iPS cell in step (a) with the epigenetic status of the embryonic stem cell.

25. The method of claim 20, wherein the candidate agent is selected from the group consisting of: a small molecule, an RNAi molecule a nucleic acid, a protein, a peptide or an antibody.

26. The method of claim 20, wherein the candidate agent alters DNA methylation status.

27. The method of claim 20, wherein, the induced pluripotent stem cell is a mammalian iPS cell.

28-62. (canceled)

63. A method for selecting an induced pluripotent stem cell (iPS), the method comprising: inhibiting DNA methylation during reprogramming of a somatic cell or cell population, wherein the reprogramming generates a population of iPS cells; and selecting an iPS cell from the population of iPS cells that has enhanced differentiation potential relative to an iPS cell generated in the absence of methylation inhibition, wherein the selected cell expresses a gene in the Dlk1-Dio3 cluster.

64. The method of claim 63, wherein the selected cell has decreased methylation of the Gtl2 gene.

65. The method of claim 63, wherein said selecting comprises selecting an iPS cell that expresses a gene in the Dlk1-Dio3 cluster from the population of iPS cells.

66. The method of claim 63, wherein inhibiting DNA methylation is effected by the inhibition of a methylase enzyme.

67. The method of claim 63, wherein the methylase enzyme is Dnmt3a.

68. The method of claim 63, wherein the inhibition of a methylase enzyme comprises inhibition of the expression of the methylase enzyme.

69. The method of claim 63, wherein said inhibition of a methylase enzyme comprises contacting said cell with an antibody or antigen-binding fragment thereof that binds said methylase enzyme.
